(19) United States
(12) Patent Application Publication
(10) Pub. No.: US 2024/0158768 A1
(43) Pub. Date: May 16, 2024

(54) ANTI-INFLAMMATORY PEPTIDES AND COMPOSITION COMPRISING THE SAME

(71) Applicants: Sang Jae KIM, Seoul (KR); GemVax & KAEL Co., Ltd., Daejeon (KR)

(72) Inventors: Sang Jae KIM, Seoul (KR); Kyung Hee KIM, Seongnam-si Gyeonggi-do (KR); Kyu-Yong LEE, Seongnam-si Gyeonggi-do (KR); Seong-Ho KOH, Seongnam-si Gyeonggi-do (KR); Bum Joon KIM, Seongnam-si Gyeonggi-do (KR); Hyun-Hee PARK, Seongnam-si Gyeonggi-do (KR); Sung Jin HUH, Seongnam-si Gyeonggi-do (KR); Woo Jin LEE, Seongnam-si Gyeonggi-do (KR); Hwain JANG, Seongnam-si Gyeonggi-do (KR); Jung Soon HA, Seongnam-si Gyeonggi-do (KR)

(21) Appl. No.: 18/411,838

(22) Filed: Jan. 12, 2024

Related U.S. Application Data
(62) Division of application No. 17/389,671, filed on Jul. 30, 2021, now Pat. No. 11,905,536.
Application No. 17/389,671 is a division of application No. 16/818,001, filed on Mar. 13, 2020, now Pat. No. 11,098,294.
Application No. 16/818,001 is a division of application No. 14/400,322, filed on Nov. 10, 2014, now abandoned.

(30) Foreign Application Priority Data
Jul. 20, 2012 (KR) 10-2012-0079096
Aug. 14, 2012 (KR) 10-2012-0089161
Aug. 14, 2012 (KR) 10-2012-0089162
Aug. 14, 2012 (KR) 10-2012-0089167
Sep. 19, 2012 (KR) 10-2012-0104144
Sep. 19, 2012 (KR) 10-2012-0104207

(51) Int. Cl. C12N 9/12; A23L 33/18; A61K 38/45

(52) U.S. Cl. CPC C12N 9/1276; A23L 33/18; A61K 38/45; A23V 2002/00; C12Y 207/07049; Y02A 50/30

(57) ABSTRACT

The present invention relates to a peptide with anti-inflammatory activity, wherein the peptide comprises any one amino acid sequence of SEQ ID NO: 1 to SEQ ID NO: 161, the peptide has above 80% homology of amino acid sequence with above-mentioned sequences, or the peptide is the fragment of the above-mentioned peptides. The present invention also relates to an inflammatory composition comprising the above mentioned peptides. According to the present invention, a peptide that has at least one amino acid sequence of SEQ ID NO: 1 to SEQ ID NO: 161 has outstanding efficacy in both suppressing inflammation and in prophylactic means. Therefore, the composition comprising the peptides of this invention can be used as anti-inflammatory pharmaceutical compositions or as cosmetic compositions, in turn, treating and preventing a variety of different types of inflammatory diseases.

### FIELD OF THE INVENTION

The present invention relates to anti-inflammatory peptides and compositions comprising the same.

### BACKGROUND OF THE INVENTION

Inflammation is a type of biological defense as a means of protecting the body from damage of biological tissues that could be caused by external physical stimuli, chemical stimuli such as exposure to various allergens, or invasion of microorganisms including bacteria, fungi and viruses.

The Cyclooxygenase(COX) pathway or Lipoxygenase(LOX) Pathway can used for signaling inflammation, which produce prostaglandin, thromboxane, etc. Once the inflammatory signal is delivered, one of many changes that happen in the body is the expansion of the blood vessel for increased blood supply around the inflammation to concentrate blood cells such as neutrophils required for the inflammatory response. However, inflammatory diseases can result if an abnormal biological defense response occurs excessively. To prevent this, drugs that suppress excessive inflammatory responses by repressing enzymes used in Inflammatory signaling pathways (for example COX-1, COX-2, 5-LOX, 12-LOX etc.) are under development.

According to response time, inflammation is categorized as acute inflammation (immediate response, non-specific response, several days to several weeks), chronic inflammation (delayed response, specific response, several weeks or more), subacute inflammation (a middle stage in between acute inflammation and chronic inflammation, characteristics of mixed product of mononuclear and polymorphounuclear).

Also, aside from peptide factors, factors such as prostaglandin, leukotriene, lipid factors including platelet activating factor (PAF), synthetic enzyme of inflammation factor, free radical such as NO (nitric oxide), many kinds of cell adhesion molecules, the immune system, and coagulation factors can cause inflammation.

Once a cell is damaged due to the known causative agents of inflammation such as external biological factors (microbes, viruses, parasites), physical factors (mechanical stimuli, heat, radiation, electricity), and chemical factors, histamine and kinin are released. The released histamine and kinin will result in angiectasis, increased capilliary permeability and concentration of macrophages at the inflammation site, and it causes increased blood flow rate, edema, immunocyte and antibody migration, pain and heat generation.

Currently used treatments for inflammation are synthetic drugs such as ibuprofen, antihistamines, steroids, cortisone, immunosuppressive agents, and immune agonist; those which only temporarily alleviate inflammation. These drugs do not fundamentally cure inflammation, and they have side effects such as hypersensitivity reaction, and deterioration of immune system,

Therefore, for effective alleviation of inflammation, research is being done to develop a substance that inhibits expression of the above mentioned inflammatory proteins. However, problems have arisen in anti-inflammation substances that had been developed previously. Diverse categories of anti-inflammatory drugs including Non-steroidal Anti-inflammatory Drugs (NSAIDs) and Steroidal Anti-inflammatory Drugs (SAIDs) have been developed; but not only do these drugs often bear side effects upon use, they also do not fundamentally cure the inflammation. Thus, there is a current need for anti-inflammatory drugs that are both physically and economically feasible. As one example, in acute or chronic inflammations such as chronic rheumatoid arthritis, not only do non-steroidal anti-inflammatory drugs suppress COX-2 enzyme activity, they are also known to suppress COX-1 activity, causing side effects such as gastrointestinal disorders.

The present invention was completed as the present inventors have found that peptides derived from telomerase can have anti-inflammatory properties.

Therefore the objective of this invention is to provide a novel peptide.

Another objective of present invention is to provide the polynucleotide that codes the novel peptide.

Another objective of present invention is to provide a peptide that has anti-inflammatory activity.

Another objective of present invention is to provide an anti-inflammatory composition that uses this peptide as an active ingredient.

Another objective of present invention is to provide a cosmetic composition that uses this peptide as an active ingredient.

Another objective of present invention is to provide a pharmaceutical composition that uses this peptide as an active ingredient.

### SUMMARY OF THE INVENTION

In one embodiment the present invention relates to a peptide with anti-inflammatory activity, wherein the peptide comprises at least one amino acid sequence of SEQ ID NO: 1 to SEQ ID NO: 161, or where the peptide has at least 80% sequence identity with the above-mentioned sequences, or the peptide is a fragment of the above-mentioned peptides.

In another embodiment, the above-mentioned fragment consists of 3 or more amino acids. For instance, the fragment may consist of 4, 5, 6, 7, 8, 9, 10, 11, 12, 13, 14, 15, 16, 17, 18, 19, 20, 21, 22, 23, 24, 25, or 26 amino acid residues.

In another embodiment, the above-mentioned peptide consists of 30 or less amino acids. For instance, the peptide may consist of 29, 28, 27, 26, 25, 24, 23, 22, 21, 20, 19, 18, 17, 16, 15, 14, 13, 12, 11, 10, 9, or 8 amino acid residues.

In another embodiment, the above-mentioned peptide consists of any one amino acid sequence of SEQ ID NO: 1 to SEQ ID NO: 161.

In another embodiment, the above-mentioned peptide comprises any one amino acid sequence selected from the group consisting of: SEQ ID NO: 1 to SEQ ID NO: 6, SEQ ID NO: 9, SEQ ID NO: 11, SEQ ID NO: 14 to 21, SEQ ID NO: 23 to SEQ ID NO: 37, SEQ ID NO: 39 to SEQ ID NO: 44, SEQ ID NO: 47 to SEQ ID NO: 53, SEQ ID NO: 55 to SEQ ID NO: 61, SEQ ID NO: 63 to SEQ ID NO: 82, SEQ ID NO: 84 to SEQ ID NO: 94, SEQ ID NO: 96, SEQ ID NO: 99 to SEQ ID NO: 104, SEQ 10 NO: 107 to SEQ ID NO: 109, SEQ ID NO: 115, SEQ ID NO: 116, SEQ ID NO: 120 to SEQ ID NO: 1.22, SEQ ID NO: 124, SEQ ID NO: 129 to SEQ ID NO: 133, SEQ ID NO: 142 to SEQ ID NO: 144, SEQ ID NO: 146, SEQ ID NO: 1.48, SEQ ID NO: 149, and SEQ ID NO: 155 to SEQ ID NO: 159.

In another embodiment, the above-mentioned peptide comprises any one amino acid sequence selected from the group consisting of: SEQ ID NO: 15 to SEQ ID NO: 18, SEQ ID NO: 23 to SEQ ID NO: 27, SEQ ID NO: 29, SEQ ID NO: 31 to SEQ ID NO: 34, SEQ ID NO: 39 to SEQ ID NO: 41, SEQ ID NO: 47, SEQ ID NO:48, SEQ ID NO: 51 to SEQ ID NO: 53, SEQ ID NO: 55 to SEQ ID NO: 58, SEQ ID NO: 61, SEQ ID NO: 65 to SEQ ID NO: 68, SEQ ID NO: 70, SEQ ID NO: 73 to SEQ ID NO: 79, SEQ ID NO: 81, SEQ ID NO: 82, SEQ ID NO: 84 to SEQ ID NO: 87, SEQ ID NO: 90 to SEQ ID NO: 94. SEQ ID NO: 96, SEQ ID NO: 99, SEQ ID NO: 101 to SEQ ID NO: 104, SEQ ID NO: 107 to SEQ ID NO: 109, SEQ ID NO: 120, SEQ ID NO: 121, SEQ ID NO: 129 to SEQ ID NO: 1.32, SEQ ID NO: 142 to SEQ ID NO: 144, SEQ ID NO: 146, SEQ ID NO: 148, SEQ ID NO: 149, and SEQ ID NO: 157 to SEQ ID NO: 159.

In another embodiment, the above-mentioned peptide comprises any one amino acid sequence selected from the group consisting of: SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 27, SEQ ID NO: 29, SEQ ID NO: 30, SEQ ID NO: 32 to SEQ ID NO: 53, SEQ ID NO: 55 to SEQ ID NO: 60, SEQ ID NO: 67, SEQ ID NO: 68, SEQ ID NO: 70, SEQ ID NO: 72 to SEQ ID NO: 82, SEQ ID NO: 84 to SEQ ID NO: 92, SEQ ID NO: 94, SEQ ID NO: 99 to SEQ ID NO: 112, SEQ ID NO: 114, SEQ ID NO: 127 to SEQ ID NO: 144, SEQ ID NO: 146, SEQ ID NO: 148, SEQ ID NO: 149, SEQ ID NO: 151 and SEQ ID NO: 153 to SEQ ID NO: 161.

In another embodiment, the above-mentioned peptide comprises any one amino acid sequence selected from the group consisting of: SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 23, SEQ ID NO: 25 to SEQ ID NO: 27, SEQ ID NO: 29, SEQ ID NO: 30, SEQ ID NO: 33 to SEQ ID NO: 43, SEQ ID NO: 156, SEQ ID NO: 157 and SEQ ID NO: 159.

In another embodiment, the above-mentioned peptide comprises any one amino acid sequence selected from the group consisting of: SEQ ID NO: 1, SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 12, SEQ ID NO: 14, SEQ ID NO: 15, SEQ ID NO: 17, SEQ ID NO: 18, SEQ ID NO: 20, SEQ 10 NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 44, SEQ ID NO: 45, SEQ ID NO: 52, SEQ ID NO: 57, SEQ ID NO: 60, SEQ ID NO: 61, SEQ ID NO: 62, SEQ ID NO: 64, SEQ ID NO: 65, SEQ ID NO: 67, SEQ ID NO: 68, SEQ ID NO: 69, SEQ ID NO: 91, SEQ ID NO: 99, SEQ ID NO: 100, SEQ ID NO: 104, SEQ ID NO: 106, SEQ ID NO: 107, SEQ ID NO: 108, SEQ ID NO: 109, SEQ ID NO: 111, SEQ ID NO: 1.12, SEQ ID NO: 115, SEQ ID NO: 117, SEQ ID NO: 118, SEQ ID NO: 119, SEQ ID NO: 120, SEQ ID NO: 122, SEQ ID NO: 124, SEQ ID NO: 125, SEQ ID NO: 126, SEQ ID NO: 129, SEQ ID NO: 1.30, SEQ ID NO: 131, SEQ ID NO: 132, SEQ ID NO: 135, SEQ ID NO: 146, SEQ ID NO: 151, SEQ ID NO: 154, and SEQ ID NO: 156.

In another embodiment, the above-mentioned peptide originates from human telomerase.

In one embodiment of the present invention, a polynucleotide encoding a peptide with anti-inflammatory activity, wherein the peptide comprises at least one amino acid sequence of SEQ ID NO: 1 to SEQ ID NO: 161, or the peptide has at least 80% sequence identity with the above-mentioned sequences, or the peptide is a fragment of the above-mentioned peptides, is provided.

In another embodiment of the polynucleotide, the above-mentioned peptide consists of 30 or less amino acids. For instance, the peptide may consist of 29, 28, 27, 26, 25, 24, 23, 22, 21, 20, 19, 18, 17, 16, 15, 14, 13, 12, 11, 10, 9, or 8 amino acid residues.

In another embodiment of the polynucleotide, the above-mentioned peptide consists of any one amino acid sequence of SEQ ID NO: 1 to SEQ ID NO: 161.

In another embodiment of the polynucleotide, the above-mentioned peptide originates from human telomerase.

In one embodiment of the present invention, anti-inflammatory composition comprising a peptide as active ingredient, wherein the peptide comprises at least one amino acid sequence of SEQ ID NO: 1 to SEQ ID NO: 161, the peptide has above 80% homology of amino acid sequence with above-mentioned sequences, or the peptide is a fragment of the above-mentioned peptides, is provided.

In another embodiment of the composition, the above-mentioned peptide consists of 30 or less amino acids, cf. above.

In another embodiment of the composition, the above-mentioned peptide consists of any one amino acid sequence of SEQ ID NO: 1 to SEQ ID NO: 161.

In another embodiment of the composition, the above-mentioned peptide originates from human telomerase.

In another embodiment of the composition, the above-mentioned composition is for treatment or prophylaxis of inflammatory disease.

In another embodiment of the composition, the above-mentioned composition is a cosmetic composition for improving or preventing skin inflammation.

In another embodiment of the composition, the above-mentioned composition is a pharmaceutical composition for treatment or prophylaxis of inflammatory diseases.

In another embodiment of the composition, the above-mentioned composition is a food composition for treatment or prophylaxis of inflammation.

In another embodiment of the composition, the above-mentioned inflammatory disease is characterized by selecting from the group consisting of (1) general or localized inflammatory disease (for example, allergies; immune-complex disease; hayfever; hypersensitive shock; endotoxin shock; cachexia, hyperthermia; granulomatosis; or sarcoidosis); (2) gastro-intestinal related diseases (for example, appendicitis; gastric ulcer; duodenal ulcer; peritonitis; pancreatitis; ulcerative, acute, or ischemic colitis; cholangitis; cholecystitis, steatorrhea, hepatitis, Crone's disease; or Whipple's Disease); (3) dermal related diseases (for example, psoriasis; burns; sunburns; dermatitis; Urticarial warts or wheal); (4) vascular related diseases (for example, angittis; vasculitis; endocarditis; arteritis; atherosclerosis; thrombophlebitis; pericarditis; congestive heart failure; myocarditis; myocardial ischemia; periarteritis nodosa; recurrent stenosis; Buerger's disease; or rheumatic fever); (5) respiratory diseases (for example, asthma; epiglottitis; bronchitis; emphysema; rhinitis; cystic fibrosis; interstitial pneumonitis; COPD (chronic obstructive pulmonary disease); adult respiratory distress syndrome; coniosis; alveolitis; bronchiolitis; pharyngitis; pleurisy; or sinusitis); (6) bone, joint, muscle and connective tissue related diseases (for example, eosinophilic granuloma; arthritis; arthralgia; osteomyelitis; dermatomyositis; fasciitis; Paget's disease; gout; periodontal disease; rheumatoid arthritis; myasthenia gravis; ankylosing spondylitis; or synovitis); (7) urogenital disorders (for example, epididymitis; vaginitis; prostatitis; or urethritis); (8) central or peripheral nervous system related diseases (for example, Alzheimer's disease; meningitis; encephalitis; multiple sclerosis; cerebral infarction; cerebral embolism; Guillain-Barre syndrome; neuritis; neuralgia; spinal cord injury; paralysis; or uveitis); (9) virus (for example, influenza; respiratory syncytial virus; HIV; hepatitis B; hepatitis C; or herpes virus), infectious disease (for example, Dengue fever; or septicemia), fungal infection (for example, candidiasis); or bacterial, parasitic, and similar microbial infection (for example, disseminated bacteremia; malaria; onchocerciasis; or amebiasis); (10) autoimmune disease (for example, thyroiditis; lupus; Goodpasture's syndrome; allograft rejection; graft versus host disease; or diabetes); and (11) cancer or tumor disease (for example, Hodgkin's disease).

In one embodiment of the present invention, a method for treating or preventing inflammatory diseases by administering the anti-inflammatory composition is provided.

In one embodiment of the present invention, a kit for prophylaxis or treatment of inflammatory diseases comprising: a peptide with anti-inflammatory activity or a composition comprising of the peptide, wherein the peptide comprises at least one amino acid sequence of SEQ ID NO: 1 to SEQ ID NO: 161, the peptide has above 80% homology with above-mentioned sequences, or the peptide is a fragment of the above-mentioned peptides; and instructions including at least one of administration dose, administration route, administration frequency, and indication of the peptide or composition, is provided.

### INDUSTRIAL APPLICABILITY

According to the present invention, a peptide that has a sequence of SEQ ID NO: 1 to SEQ ID NO: 161 has outstanding efficacy in both suppressing inflammation and in prophylactic means. Therefore, the composition comprising the peptides of this invention can be used as anti-inflammatory pharmaceutical composition or as cosmetic composition, in turn, treating and preventing a variety of different types of inflammatory diseases.

### REFERENCES

KR2012-0130996A
KR2012-0133661A
KR2011-0060940A
US2011-0150873A1
Bonaldi T et al., EMBO 3, (22)5551-60, 2003
Yankner B A et al, Science (New York, N.Y.) [1990, 250(4978):279-282]
Dahigren K N et al, J. Biol. Chem. 277:32046-32053, 2002.

### BRIEF DESCRIPTION OF THE DRAWINGS

FIG. 1 to FIG. 16 are results from screening TNF-α inhibition effects on monocytes.

FIG. 17 to FIG. 35 are results from screening TNF-α inhibition effects on cell line THP-1.

FIG. 36 to FIG. 108 are the western blot results of selected peptides which showed accumulation of HMGB1 In the cell.

### DETAILED DESCRIPTION OF THE INVENTION

Since the present invention can have adaptability for diverse transformation and examples of practical application, below is a more detailed description of the present invention. Nevertheless, this is no means to limit the form of practical application; it should be understood that the intention is to include the concept and the extent of technology in all of the transformation, equivalents to alternatives. In describing the present invention, if any detailed description about the prior art is considered to deteriorate the fundamental principles of the present invention, the description will be omitted.

A telomere is known as a repetitive sequence of genetic material at the ends of chromosomes that prevent chromosomes from damage or merging of other chromosomes. The length of a telomere is shortened at each cell division, and after a certain number of cell division, the telomere length is extremely shortened to the extent in which the cell stops dividing and dies. On the other hand, the elongation of telomeres is known to extend the life span of a cell. For an example, cancer cells excrete an enzyme called telomerase, which prevents shortening of telomeres, thus resulting in proliferation of cancer cells. The present invention was accomplished upon the discovery of telomerase-derived peptides with anti-inflammatory effects.

In one embodiment of the present invention, a peptide with anti-inflammatory activities is provided. The peptide comprises at least one amino acid sequence of SEQ ID NO: 1 to SEQ ID NO: 161, the peptide has above 80% homology with above-mentioned sequences, or the peptide is a fragment of the above-mentioned peptides.

The peptides described in SEQ ID NO: 1 to SEQ ID NO: 161 are as the following table 1. SEQ ID NO: 162 lists the order of full length of human telomerase protein. SEQ ID NO: 163 lists the telomerase-derived peptide that consists of 16 amino acid sequence.

The “name” in Table 1 below was used for distinction of peptides. In a different specific embodiment of the present invention, more than one peptide of the mentioned peptides in SEQ ID NO: 1 to SEQ ID NO: 161 comprise a “synthetic peptide”, a synthesized peptide of selected areas of the telomerase. In the present specification, the term “pep” herein relates to a peptide that has any one of the SEQ ID NO: 1 to SEQ ID NO: 161, or, a peptide comprising a amino acid sequence above 80% homology with above mentioned sequences, or a peptide fragment of above-mentioned peptides.

| | | POSITION IN | | |
|---|---|---|---|---|
| SEQ ID NO | NAME | TELOMERASE | SEQUENCE | LENGTH |
| 1. | pep2 | [660-689] | ALFSVLNYERARRPGLLGASVLGLDDIHRA | 30 aa |
| 2. | pep3 | [663-677] | SVLNYERARRPGLG | 15 aa |
| 3. | pep4 | [674-683] | GLLSASVLGL | 10 aa |
| 4. | pep5 | [615-624] | ALLTSRLRFI | 10 aa |
| 5. | pep6 | [613-621] | RPALLTSRL | 9 aa |
| 6. | pep7 | [653-661] | RLTSRVKAL | 9 aa |
| 7. | pep8 | [691-705] | RTFVLRVRAQDPPPE | 15 aa |
| 8. | pep9 | [653-667] | RLTSRVKALFSVLNY | 15 aa |
| 9. | pep10 | [651-665] | AERLTSRVKALFSVL | 15 aa |
| 10. | pep11 | [667-675] | YERARRPGL | 9 aa |
| 11. | pep12 | [675-683] | LLGASVLGL | 9 aa |
| 12. | pep13 | [680-689] | VLGLDDIHRA | 10 aa |
| 13. | pep14 | [677-686] | GASVLGLDDI | 10 aa |
| 14. | pep15 | [660-669] | ALFSVLNYER | 10 aa |
| 15. | pep16 | [663-672] | SVLNYERARR | 10 aa |
| 16. | pep17 | [679-688] | SVLGLDDIHR | 10 aa |
| 17. | pep18 | [662-671] | FSVLNYERAR | 10 aa |
| 18. | pep19 | [666-675] | NYERARRPGL | 10 aa |
| 19. | pep20 | [667-676] | YERARRPGLL | 10 aa |
| 20. | pep21 | [672-681] | RPGLLGASVL | 10 aa |
| 21. | pep22 | [668-676] | ERARRPGLL | 9 aa |
| 22. | pep23 | [680-688] | VLGLDDIHR | 9 aa |
| 23. | pep24 | [663-671] | SVLNYERAR | 9 aa |
| 24. | pep25 | [664-672] | VLNYERARR | 9 aa |
| 25. | pep26 | [670-678] | ARRPGLLGA | 9 aa |
| 26. | pep27 | [673-681] | PGLLGASVL | 9 aa |
| 27 | pep28 | [671-679] | RRPGLLGAS | 9 aa |
| 28. | pep29 | [660-668] | ALFSVLNYE | 9 aa |
| 29. | pep30 | [674-682] | GLLGASVLG | 9 aa |
| 30. | pep31 | [679-687] | SVLGLDDIH | 9 aa |
| 31. | pep32 | [668-675] | ERARRPGL | 8 aa |
| 32. | pep33 | [670-677] | ARRPGLLG | 8 aa |
| 33. | pep34 | [674-681] | GLLGASVL | 8 aa |
| 34. | pep35 | [669-676] | RARRPGLL | 8 aa |
| 35. | pep36 | [676-683] | LGASVLGL | 8 aa |
| 36. | pep37 | [563-577] | VTETTFQKNRLFFYR | 15 aa |
| 37. | pep38 | [573-587] | LFFYRKSVWSKLQSI | 15 aa |
| 38. | pep39 | [583-597] | KLQSIGIRQHLKRVQ | 15 aa |
| 39. | pep40 | [603-617] | EAEVRQHREARPALL | 15 aa |
| 40. | pep41 | [613-627] | RPALLTSRLRFIPKP | 15 aa |
| 41. | pep42 | [623-637] | FIPKPDGLRPIVNMD | 15 aa |
| 42. | pep43 | [643-657] | RTFRREKRAERLTSR | 15 aa |
| 43. | pep45 | [683-697] | LDDIHRAWRTFVLRV | 15 aa |
| 44. | pep46 | [693-707] | FVLRVRAQDPPPELY | 15 aa |
| 45. | pep47 | [721-735] | PQDRLTEVIASIIKP | 15 aa |
| 46. | pep48 | [578-592] | KSVWSKLQSIGIRQH | 15 aa |
| 47. | pep49 | [593-608] | LKRVQLRELSEAEVRQ | 16 aa |
| 48. | pep50 | [1-20] | MPRAPRCRAVRSLLRSHYRE | 20 aa |
| 49. | pep51 | [21-40] | VLPLATFVRRLGPQGWRLVQ | 20 aa |
| 50. | pep52 | [41-60] | RGDPAAFRALVAQCLVCVPW | 20 aa |
| 51. | pep53 | [61-80] | DARPPPAAPSFRQVSCLKEL | 20 aa |
| 52. | pep54 | [81-100] | VARVLQRLCERGAKNVLAFG | 20 aa |
| 53. | pep55 | [101-120] | FALLDGARGGPPEAFTTSVR | 20 aa |
| 54. | pep56 | [121-140] | SYLPNTVTDALRGSGAWGLL | 20 aa |
| 55. | pep57 | [141-160] | LRRVGDDVLVHLLARCALFV | 20 aa |
| 56. | pep58 | [161-180] | LVAPSCAYQVCGPPLYQLGA | 20 aa |
| 57. | pep59 | [181-200] | ATQARPPPHASGPRRRLGCE | 20 aa |
| 58. | pep60 | [201-220] | RAWNHSVREAGVPLGPLAPG | 20 aa |
| 59. | pep61 | [221-240] | ARRRGGSASRSLPLPKRPRR | 20 aa |
| 60. | pep62 | [241-260] | GAAPEPERTPVGQGSWAHPG | 20 aa |
| 61. | pep63 | [261-280] | RTRGPSDRGFCVVSPARPAE | 20 aa |
| 62. | pep64 | [281-300] | EATSLEGALSGTRHSHPSVG | 20 aa |
| 63. | pep65 | [301-320] | RQHHAGPPSTSRPPRPWDTP | 20 aa |
| 64. | pep66 | [321-340] | CPPVYAETKHFLYSSGDKEQ | 20 aa |
| 65. | pep67 | [341-360] | LRPSFLLSSLRPSLTGARRL | 20 aa |
| 66. | pep68 | [361-380] | VETIFLGSRPWMPGTPRRLP | 20 aa |
| 67. | pep69 | [381-400] | RLPQRYWQMRPLFLELLGNH | 20 aa |
| 68. | pep70 | [401-420] | AQCPYGVLLKTHCPLRAAVT | 20 aa |
| 69 | pep71 | [421-440] | PAAGVCAREKPQGSVAAPEE | 20 aa |
| 70. | pep72 | [441-460] | EDTDPRRLVQLLRQHSSPWQ | 20 aa |
| 71. | pep73 | [461-480] | VYGFVRACLRRLVPPGLWGS | 20 aa |
| 72. | pep74 | [481-500] | RHNERRFLRNTKKFISLGKH | 20 aa |
| 73. | pep75 | [501-520] | AKLSLQELTWKMSVRDCAWL | 20 aa |
| 74. | pep76 | [521-540] | RRSPGVGCVPAAEHRLREEI | 20 aa |
| 75. | pep77 | [541-560] | LAKFLHWLMSVYVVELLRSF | 20 aa |
| 76. | pep78 | [561-580] | FYVTETTFQKNRLFFYRKSV | 20 aa |
| 77. | pep79 | [581-600] | WSKLQSIGIRQHLKRVQLRE | 20 aa |
| 78. | pep80 | [601-620] | LSEAEVRQHREARPALLTSR | 20 aa |
| 79. | pep81 | [621-640] | LRFIPKPDGLRPIVNMDYVV | 20 aa |
| 80. | pep82 | [641-660] | GARTFRREKRAERLTSRVKA | 20 aa |
| 81. | pep83 | [661-680] | LFSVLNYERARRPGLLGASV | 20 aa |
| 82. | pep84 | [681-700] | LGLDDIHRAWRTFVLRVRAQ | 20 aa |
| 83. | pep85 | [701-720] | DPPPELYFVKVDVTGAYDTI | 20 aa |
| 84. | pep86 | [721-740] | PQDRLTEVIASIIKPQNTYC | 20 aa |
| 85. | pep87 | [741-760] | VRRYAVVQKAAHGHVRKAFK | 20 aa |
| 86. | pep88 | [761-780] | SHVSTLTDLQPYMRQFVAHL | 20 aa |
| 87. | pep89 | [781-800] | QETSPLRDAVVIEQSSSLNE | 20 aa |
| 88. | pep90 | [801-820] | ASSGLFDVFLRFMCHHAVRI | 20 aa |
| 89. | pep91 | [821-840] | RGKSYVQCQGIPQGSILSTL | 20 aa |
| 90. | pep92 | [841-860] | LCSLCYGDMENKLFAGIRRD | 20 aa |
| 91. | pep93 | [861-880] | GLLLRLVDDFLLVTPHLTHA | 20 aa |
| 92. | pep94 | [881-900] | KTFLRTLVRGVPEYGCVVNL | 20 aa |
| 93. | pep95 | [901-920] | RKTVVNFPVEDEALGGTAFV | 20 aa |
| 94. | pep96 | [921-940] | QMPAHGLFPWCGLLLDTRTL | 20 aa |
| 95. | pep97 | [941-960] | EVQSDYSSYARTSIRASLTF | 20 aa |
| 96. | pep98 | [961-980] | NRGFKAGRNMRRKLFGVLRL | 20 aa |
| 97. | pep99 | [981-1000] | KCHSLFLDLQVNSLQTVCTN | 20 aa |
| 98. | pep100 | [1001-1020] | IYKILLLQAYRFHACVLQLP | 20 aa |
| 99. | pep101 | [1021-1040] | FHQQVWKNPTFFLRVISDTA | 20 aa |
| 100. | pep102 | [1041-1060] | SLCYSILKAKNAGMSLGAKG | 20 aa |
| 101. | pep103 | [1061-1080] | AAGPLPSEAVQWLCHQAFLL | 20 aa |
| 102. | pep104 | [1081-1100] | KLTRHRVTYVPLLGSLRTAQ | 20 aa |
| 103 | pep105 | [1101-1120] | TQLSRKLPGTTLTALEAAAN | 20 aa |
| 104. | pep106 | [1121-1132] | PALPSDFKTILD | 20 aa |
| 105. | pep107 | [1-10] | MPRAPRCRAV | 20 aa |
| 106. | pep108 | [11-30] | RSLLRSHYREVLPLATFVRR | 20 aa |
| 107. | pep109 | [31-50] | LGPQGWRLVQRGDPAAFRAL | 20 aa |
| 108. | pep110 | [51-70] | VAQCLVCVPWDARPPPAAPS | 20 aa |
| 109. | pep110 | [71-90] | FRQVSCLKELVARVLQRLCE | 20 aa |
| 110. | pep112 | [91-110] | RGAKNVLAFGFALLDGARGG | 20 aa |
| 111. | pep113 | [111-130] | PPEAFTTSVRSYLPNTVTDA | 20 aa |
| 112. | pep114 | [131-150] | LRGSGAWGLLLRRVGDDVLV | 20 aa |
| 113. | pep115 | [151-170] | HLLARCALFVLVAPSCAYQV | 20 aa |
| 114. | pep116 | [171-190] | CGPPLYQLGAATQARPPPHA | 20 aa |
| 115. | pep117 | [191-210] | SGPRRRLGCERAWNHSVREA | 20 aa |
| 116. | pep118 | [211-230] | GVPLGLPAPGARRRGGSASR | 20 aa |
| 117 | pep119 | [231-250] | SLPLPKRPRRGAAPEPERTP | 20 aa |
| 118. | pep120 | [251-270] | VGQGSWAHPGRTRGPSDRGF | 20 aa |
| 119. | pep121 | [271-290] | CVVSPARPAEEATSLEGALS | 20 aa |
| 120. | pep122 | [291-310] | GTRHSHPSVGRQHHAGPPST | 20 aa |
| 121. | pep123 | [311-330] | SRPPRPWDTPCPPVYAETKH | 20 aa |
| 122. | pep124 | [331-350] | FLYSSGDKEQLRPSFLLSSL | 20 aa |
| 123. | pep125 | [351-370] | RPSLTGARRLVETIFLGSRP | 20 aa |
| 124. | pep126 | [371-390] | WVPGTPRRLPRLPQRYWQMR | 20 aa |
| 125. | pep127 | [391-410] | PLFLELLGNHAQCPYGVLLK | 20 aa |
| 126. | pep128 | [411-430] | THCPLRAAVTPAAGVCAREK | 20 aa |
| 127. | pep129 | [431-450] | PQGSVAAPEEEDTDPRRLVQ | 20 aa |
| 128. | pep130 | [451-470] | LLRQHSSPWQVYGFVRACLR | 20 aa |
| 129. | pep131 | [471-490] | RLVPPGLWGSHRNERRFLRN | 20 aa |
| 130. | pep132 | [491-510] | TKKFISLGKHAKLSLQELTW | 20 aa |
| 131. | pep133 | [511-530] | KMSVRDCAWLRRSPGVGCVP | 20 aa |
| 132. | pep134 | [531-550] | AAEHRLREEILAKFLHWLMS | 20 aa |
| 133. | pep135 | [551-570] | VYVVELLRSFFYVTETTFQK | 20 aa |
| 134. | pep136 | [571-590] | NRLFFYRKSVWSKLQSIGIR | 20 aa |
| 135. | pep137 | [591-610] | QHLKRVQLRELSEAEVRQHR | 20 aa |
| 136. | pep138 | [611-630] | EARPALLTSRLRFIPKPDGL | 20 aa |
| 137. | pep139 | [631-650] | RPIVNMDYVVGARTFRREKR | 20 aa |
| 138. | pep140 | [651-670] | AERLTSRVKALFSVLNYERA | 20 aa |
| 139. | pep141 | [671-690] | RRPGLLGASVLGLDDIHRAW | 20 aa |
| 140. | pep142 | [691-710] | RTFVLRVRAQDPPPELYFVK | 20 aa |
| 141. | pep143 | [711-730] | VDVTGAYDTIPQDRLTEVIA | 20 aa |
| 142. | pep144 | [731-750] | SIIKPQNTCYVRRYAVVQKA | 20 aa |
| 143. | pep145 | [751-770] | AHGHVRKAFKSHVSTLTDLQ | 20 aa |
| 144. | pep146 | [771-790] | PYMRQFVAHLQETSPLRDAV | 20 aa |
| 145. | pep147 | [791-810] | VIEQSSSLNEASSGLFDVFL | 20 aa |
| 146. | pep148 | [811-830] | RFMCHHAVRIRGKSYVQCQC | 20 aa |
| 147. | pep149 | [831-850] | IPQGSILSTLLCSLCYGDME | 20 aa |
| 148. | pep150 | [851 870] | NKLFAGIRRDGLLLRLVDDF | 20 aa |
| 149. | pep151 | [871-890] | LLVTPHLTHAKTFLRTLVRG | 20 aa |
| 150. | pep152 | [891-910] | VPEYGCVVNLRKTVVNFPVE | 20 aa |
| 151. | pep153 | [911-930] | DEALGGTAFVQMPAHGLFPW | 20 aa |
| 152. | pep154 | [931-950] | CGLLLDTRTLEVQSDYSSYA | 20 aa |
| 153. | pep155 | [951-970] | RTSIRASLTFNRGFKAGRNM | 20 aa |
| 154. | pep156 | [971-990] | RRKLPGVLRLKCHSLFLDLQ | 20 aa |
| 155. | pep157 | [991-1010] | VNSLQTVCTNIYKILLLQAY | 20 aa |
| 156. | pep158 | [1011-1030] | RFHACVLQLPFHQQVWKNPT | 20 aa |
| 157. | pep159 | (1031-1050] | FFLRVISDTASLCYSILAKA | 20 aa |
| 158. | pep160 | [1051-1070] | NAGMSLGAKGAAGPLPSEAV | 20 aa |
| 159 | pep161 | [1071-1090] | QWLCHQAFLLKLTRHRVTYV | 20 aa |
| 160. | pep162 | [1091-1110] | PLLGSLRTAQTQLSRKLPGT | 20 aa |
| 161. | pep163 | [111-1132] | TLTALEAAANPALPSDFKTILD | 20 aa |
| 162. | Telomerase | [1-1132] | MPRAPRCRAVRSLLRSHYREVLPLATFVRR | 1132 aa |
| | | | LGPQGWRLVQRGDPAAFRALVAQCLVCVPW | |
| | | | DARPPPAAPSFRQVSVLKELVARVLQRLCERG | |
| | | | AKNVLAFGFALLDGARGGPPEAFTTSVRSYLP | |
| | | | NTVTDALRGSGAWGLLLRRVGDDVLVHLLAR | |
| | | | CALFVLVAPSCAYQVCGPPLYQLGAATQARPP | |
| | | | PHASGPRRRLGCERSWNHSVREAGVPLGLPA | |
| | | | PGARRRGGSASRSLPLPKRPRRGAAPEPERTP | |
| | | | VGQGSWAHPGRTRGPSDRGFCVVSPARPAE | |
| | | | EATSLEGALSGTRHSHPSVGRQHHAGPPSTS | |
| | | | RPPRPWDTPCPPVYAETKHFLYSSGDKEQLR | |
| | | | PSFLLSSLRPSLTGARRLVETIFLGSRPWMPG | |
| | | | TPRRLPRLPQRYWQMRPLFLELLGNHAQCPY | |
| | | | GVLLKTHCPLRAAVTPAAGVCAREKPQGSVA | |
| | | | APEEEDTDPRRLVQLLRQHSSPWQVYGFVRA | |
| | | | CLRRLVPPGLWGSRHNERRFLRNTKKFISLG | |
| | | | KHAKLSLQELTWKMSVRDCAWLRRSPGVGC | |
| | | | VPAAEHRLREEILAKFLHWLMSVYVVELLRSF | |
| | | | FYVTETTFQKNRLFFYRKSVWSKLQSIGIRQH | |
| | | | LKRVQLRELSEAEVRQHREARPALLTSRLRFI | |
| | | | PKPDGLRPIVNMDYVVGARTFRREKRAERLT | |
| | | | SRVKALFSVLNYERARRPGLLGASVLGLDDIH | |
| | | | FAWRTFVLRVRAQDPPPELYFVKVDVTGAYD | |
| | | | TIPQDRLTEVIASIIKPQNTYCVRRYAVVQKA | |
| | | | AHGHVRKAFKSHVSTLDTDLQPYMRQFVAHLQ | |
| | | | ETSPLRDAVVIEQSSSLNEASSGLFDVFLRFM | |
| | | | CHHAVRIRGKSYVQCQGIPQGSILSTLLCSLC | |
| | | | YGDMENKLFAGIRRDGLLLRLVDDFLLVTPHL | |
| | | | THAKTFLRTLVRGVPEYGCVVNLRKTVVNFPV | |
| | | | EDEALGGTAFVQMPAHGLFPWCGLLLDTRTL | |
| | | | EVQSDYSSYARTSIRASLTFNRGFKAGRNMR | |
| | | | RKLFGVLRLKCHSLFLDLQVNSLQTVCTNIYK | |
| | | | ILLLQAYRFHACVLQLPFHQQVWKNPTFFLRV | |
| | | | ISDTASLCYSILKAKNAGMSLGAKGAAGPLPS | |
| | | | EAWQWLCHQAFLLKLTRHRVTYVPLLGSLRTA | |
| | | | QTQLSRKLPGTTLTALEAAANPALPSDFKTIL | |
| | | | D | |
| 163. | pep 1 | [611-626] | EARPALLTSRLRFIPK | 16 aa |

In one embodiment of the present invention, a polynucleotide that codes a peptide with anti-inflammatory activities is provided. The polynucleotide codes a peptide comprising at least one amino acid sequence of SEQ ID NO: 1 to SEQ ID NO: 161, a peptide having above 80% homology with above-mentioned sequences, or a peptide being a fragment of the above-mentioned peptides. The polynucleotide mentioned above enables production of the peptides in large quantities. For example, cultivation of vectors that include polynucleotides encoding peptides allows production of peptides in large quantities.

The peptides disclosed herein can include a peptide comprising amino acid sequence above 80%, above 85%, above 90%, above 95%, above 96%, above 97%, above 98%, or above 99% homology. Moreover, the peptides disclosed in the present invention can include a peptide comprising SEQ ID NO: 1 or its fragments, and a peptide with more than 1 transformed amino acid, more than 2 transformed amino acid, more than 3 transformed amino acid, more than 4 transformed amino acid, more than 5 transformed amino acid, more than 6 transformed amino acid, or more than 7 transformed amino acid.

In the present specification and claims, the terms “homology” and “sequence identity” are used interchangeably to indicate the degree of sequence overlap between two amino acid (or if relevant: nucleic acid) sequences.

Unless otherwise stated the term “Sequence identity” for peptides as used herein refers to the sequence identity calculated as (n_{ref}−n_{dif})·100/n_{ref}, wherein n_{dif} is the total number of non-identical residues in the two sequences when aligned so that a maximum number of amino acids are identical and wherein n_{ref} is the number of residues in the shortest of the sequences. Hence, the DNA sequence agtcagtc will have a sequence identity of 75% with the sequence aatcaatc (n_{dif}=2 and n_{ref}=8).

In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

In one embodiment of the present invention, changes in amino acid sequence belong to the modification of peptide's physical and chemical characteristics. For example, amino acid transformation can be performed by improving thermal stability of the peptide, altering substrate specificity, and changing the optimal pH.

In one embodiment of the present invention, a peptide comprising amino acid sequence of at least one of SEQ ID NO: 1 to SEQ ID NO: 161, a peptide comprising of amino acid sequence above 80% homology with above-mentioned sequences, or a peptide fragment of above-mentioned peptides is preferably made of 30 or less amino acids.

In one embodiment of the present invention, a peptide comprising amino acid sequence of at least one of SEQ ID NO: 1 to SEQ ID NO: 161, a peptide comprising of amino acid sequence above 80% homology with above-mentioned sequences, or a peptide fragment of above-mentioned peptides comprises a peptide originages from telomerase, more specifically, telomerase of Homo sapiens.

The term “amino acid” herein includes not only the 22 standard amino acids that are naturally integrated into peptide but also the D-isomers and transformed amino acids. Therefore, in a specific embodiment of the present invention, a peptide herein includes a peptide having D-amino acids. On the other hand, a peptide may include non-standard amino acids such as those that have been post-translationally modified. Examples of post-translational modification include phosphorylation, glycosylation, acylation (including acetylation, myristoylation, plamitoylation), alkylation, carboxylation, hydroxylation, glycation, biotinylation, ubiquitinylation, transformation in chemical properties (e.g. β-removing deimidation, deamidation) and structural transformation (e.g. formation or disulfide bridge). Also, changes of amino acids are included and the changes of amino acids occur due to chemical reaction during the combination process with crosslinkers for formation of a peptide conjugate.

A peptide disclosed herein may be a wild-type peptide that has been identified and isolated from natural sources. On the other hand, when compared to peptide fragments of SEQ ID NO: 1, the peptides disclosed herein may be artificial mutants that comprise one or more substituted, deleted and/or inserted amino acids. Amino acid alteration in wild-type polypeptide—not only in artificial mutants comprises conservative substitution of amino acids that do not influence protein folding and or activation. Examples of conservative substitution belong to the group consisting of basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). The amino acid substitutions that do not generally alter the specific activity are known in the art of the present invention. Most common occurred alteration are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly, and the opposite alterations. Another example of conservative substitutions are shown in the following table 2.

TABLE 2

| Original | | Preferable residue |
|---|---|---|
| amino acid | Examples of residue substitution | substitution |
| Ala (A) | val; leu; Ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | ala | Ala |
| His (H) | asn; gln; lys; arg | Arg |
| Ile (I) | leu; val; met; ala; phe; norleucine | Leu |
| Leu (L) | norleucine; Ile; val; met; ala; phe | Ile |
| Lys (K) | arg; gln; asn | Arg |
| Met (M) | leu; phe; Ile | Leu |
| Phe (F) | leu; val; Ile; ala; tyr | Tyr |
| Pro (P) | ala | Ala |
| Ser (S) | thr | Thr |
| Thr (T) | ser | Ser |
| Trp (W) | tyr; phe | Tyr |
| Tyr (Y) | trp; phe; thr; ser | Phe |
| Val (V) | Ile; leu; met; phe; ala; norleucine | Leu |

The substantial transformation of the biological properties of peptides are performed by selecting significantly different substitution in the following efficacies: (a) the efficacy in maintaining the structure of the polypeptide backbone in the area of substitution, such as sheet or helical three-dimensional structures, (b) the efficacy in maintaining electrical charge or hydrophobicity of the molecule in the target area, or (c) the efficacy of maintaining the bulk of the side chain. Natural residues are divided into groups by general side chain properties as the following:
(1) hydrophobicity: Norleucine, met, ala, val, leu, ile;
(2) neutral hydrophilicity: cys, ser, thr;
(3) acidity: asp, glu;
(4) basicity: asn, gin, his, lys arg;
(5) residue that affects chain orientation: gly, pro; and
(6) aromaticity: trp, tyr, phe.

Non-conservative substitutions may be performed by exchanging a member of the above classes to that of a different class. Any cysteine residues that are not related in maintaining the proper three-dimensional structure of the peptide can typically be substituted into serine, thus increasing the oxidative stability of the molecule and preventing improper crosslinkage, Conversely, improvement of stability can be achieved by adding cysteine bond(s) to the peptide.

Altered types of amino acids variants of peptides are those that antibody glycosylation pattern changed. The term “change” herein relates to deletion of carbohydrate residues and/or addition of at least one glycosylated residues that do not exist within a peptide.

Glycosylation in peptides are typically N-connected or O-connected. The term “N-connected” herein relates to that carbohydrate residues are attached to the side chain of asparagine residues. As tripeptide sequences, asparagine-X-serine and asparagine-X-threonine (where the X is any amino acid except proline) are the recognition sequence for attaching carbohydrate residue enzymatically to the side chain of asparagine. Therefore, with the presence of one of these tripeptide sequences in a polypeptide, the potential glycosylation sites are created. “O-connected glycosylation” means attaching one or sugar N-acetylgalactosamine, galactose, or xylose to hydroxyl amino acids. The hydroxyl amino acids are most typically serine or threonine, but 5-hydroxyproline or 5-hydroxylysine can be used.

Addition of glycosylation site to a peptide is conveniently performed by changing amino acid sequence to contain tripeptide sequence mentioned above (for N-linked glycosylation sites). These changes may be made by addition of at least one serine or theronine residues to the first antibody sequence, or by substitution with those residues (for O-linked glycosylation sites).

In one embodiment of the present invention, a polynucleotide is a nucleic acid molecule that can be spontaneous or artificial DNA or RNA molecules, either single-stranded or double-stranded. The nucleic acid molecule can be one or more nucleic acids of same type (for example, having a same nucleotide sequence) or nucleic acids of different types. The nucleic acid molecules comprise one or more DNA, cDNA, decoy DNA, RNA, siRNA, miRNA shRNA, stRNA, snoRNA, snRNA PNA, antisense oligomer, plasmid and other modified nucleic acids, but not limited to those.

A HMGB1 protein is known as a cytokine. It first undergoes acetylation and translocation to cytoplasm by external stimulation. Then it is secreted out of the cell, therefore serving the role of inflammation-causing cytokine. Because when one has an inflammation due to such activity, HMGB1 protein is secreted out of the cell, and patients with inflammatory diseases such as Churg strauss syndrome, rheumatoid arthritis and Sjogren's syndrome will present with elevated serum levels of HMGB1. Hence, if nucleus contains large amount of HMGB1 even when there is a stimulus that causes inflammation, it is suggestive of the fact that HMGB1 is not being secreted out of the cell, which means inflammation is being suppressed.

In one embodiment of the present invention, when treated a cell with a peptide comprising any one amino acid sequence of SEQ ID NO: 1 to SEQ ID NO: 16.1, a peptide having above 80% homology of amino acid sequence with above-mentioned sequences, or a fragment of the above-mentioned peptides, amount of HMGB1 within the nucleus increases. This represents that the peptides mentioned above have excellent inflammation preventive or suppressive effects.

Also, in specific embodiments of the present invention, a peptide comprising any one amino acid sequence of SEQ ID NO: 1 to SEQ ID NO: 161, a peptide having above 80% homology of amino acid sequence with above-mentioned sequences, or a fragment of the above-mentioned peptides, has an advantage on that it has high feasibility due to its low toxicity within a cell.

In the present invention, an “inflammatory disease” is a broad indication that refers to any disease that designates inflammation as a main cause or inflammation caused by disease. Specifically, an inflammatory disease includes (1) general or localized inflammatory disease (for example, allergies; immune-complex disease; hayfever; hypersensitive shock; endotoxin shock; cachexia, hyperthermia; granulomatosis; or sarcoidosis); (2) gastro-intestinal related diseases (for example, appendicitis; gastric ulcer; duodenal ulcer; peritonitis; pancreatitis; ulcerative, acute, or ischemic colitis; cholangitis; cholecystitis, steatorrhea, hepatitis, Crone's disease; or Whipple's Disease); (3) dermal related diseases (for example, psoriasis; burns; sunburns; dermatitis; Urticarial warts or wheal); (4) vascular related diseases (for example, anglitis; vasculitis; endocarditis; arteritis; atherosclerosis; thrombophlebitis; pericarditis; congestive heart failure; myocarditis; myocardial ischemia; periarteritis nodosa; recurrent stenosis; Buerger's disease; or rheumatic fever); (5) respiratory diseases (for example, asthma; epiglottitis; bronchitis; emphysema; rhinitis; cystic fibrosis; interstitial pneumonitis; COPD (chronic obstructive pulmonary disease); adult respiratory distress syndrome; coniosis; alveolitis; bronchiolitis; pharyngitis; pleurisy; or sinusitis); (6) bone, joint, muscle and connective tissue related diseases (for example, eosinophilic granuloma; arthritis; arthralgia; osteomyelitis; dermatomyositis; fasciitis; Pager's disease; gout; periodontal disease; rheumatoid arthritis; myasthenia gravis; ankylosing spondylitis; or synovitis); (7) urogenital disorders (for example, epididymitis; vaginitis; prostatitis; or urethritis); (8) central or peripheral nervous system related diseases (for example, Alzheimer's disease: meningitis; encephalitis; multiple sclerosis; cerebral infarction; cerebral embolism; Guillain-Barre syndrome; neuritis; neuralgia; spinal cord injury; paralysis; or uveitis); (9) virus (for example, influenza; respiratory syncytial virus; HIV; hepatitis 8; hepatitis C; or herpes virus), infectious disease (for example, Dengue fever; or septicemia), fungal infection (for example, candidiasis); or bacterial, parasitic, and similar microbial infection (for example, disseminated bacteremia; malaria; onchocerciasis; or amebiasis); (10) autoimmune disease (for example, thyroiditis; lupus; Goodpasture's syndrome; allograft rejection; graft versus host disease; or diabetes); and (11) cancer or tumor disease (for example, Hodgkin's disease), but not limited to those.

Treating the inflammatory component of such diseases has been a major goal of the global pharmaceutical industry for a number of decades, and a wide variety of useful treatments have been developed. Examples include the corticosteroids (a range of natural, semisynthetic and synthetic agents designed to mimic the effect of cortisol, including prednisolone, methylprednisolone, dexamethasone, betamethasone, fluticasone and so forth), cyclooxygenase inhibitors (both non-selective or cox-1 selective, such as indomethacin, sulfasalazine and aspirin, and more recently cox-2 selective, such as celecoxib), leukotriene blockers (such as monteleukast) and anti-TNFs (such as modified monoclonal neutralising antibodies, including infliximab (Remicade™) and adalimumab (Humira™), TNF receptor fusion proteins, such as etanercept (Enbrel™), as well as small molecule TNF-α synthesis inhibitors like thalidomide).

In one embodiment of the present invention, an anti-inflammatory composition comprising a peptide as an active ingredient is provided. The peptide comprises at least one amino acid sequence of SEQ ID NO: 1 to SEQ 10 NO: 161, the peptide has above 80% homology with above-mentioned sequences, or the peptide is a fragment of the above-mentioned peptides.

In one embodiment of the present invention, the anti-Inflammatory composition may contain 0.1 μg/mg to 1 mg/mg, specifically 1 μg/mg to 0.5 mg/mg, more specifically 10 μg/mg to 0.1 mg/mg of a peptide comprising of amino acid sequence of at least one of SEQ ID NO: 1 to SEQ ID NO: 161, a peptide comprising of amino acid sequence above 80% homology with above-mentioned sequences, or peptide fragment of above-mentioned peptides. When the peptide is contained in the above mentioned range, all the safety and stability of the composition may be satisfied and appropriate in terms of cost-effectiveness.

In one embodiment of the present invention, the composition may have application with all animals including human, dog, chicken, pig, cow, sheep, guinea pig, and monkey.

In one embodiment of the present invention, the medical composition for the use of treatment or prophylaxis of inflammatory disease with an active ingredient that is comprised of a peptide consisting of an amino acid sequence from SEQ ID NO: 1 to SEQ ID NO: 161, a peptide comprising of amino acid sequence above 80% homology with above-mentioned sequences, or peptide fragment of SEQ ID NO:1, is provided. In one embodiment of the present invention, the pharmaceutical composition may be administered through oral, rectal, transdermal, intravenous, intramuscular, intraperitoneal, in the bone marrow, epidural or subcutaneous means.

Forms of oral administration may be, but not limited to, tablets, pills, soft or hard capsules, granules, powders, solution, or emulsion. Forms of non-oral administration may be, but not limited to, injections, drips, lotions, ointments, gels, creams, suspensions, emulsions, suppository, patch, or spray.

In one embodiment of the present invention, the pharmaceutical composition, if necessary, may contain additives, such as diluents, excipients, lubricants, binders, disintegrants, buffers, dispersants, surfactants, coloring agents, aromatics or sweeteners. In one embodiment of the present invention, the pharmaceutical composition can be manufactured by conventional methods of the industry in the art.

In one embodiment of the present invention, the active ingredient of the medical composition may vary according to the patient's age, sex, weight, pathology and state, administration route, or prescriber's judgment. Dosage based on these factors is determined within levels of those skilled in the art, and the daily dose for example may be, but not limited to, 0.1 μg/kg/day to 1 g/kg/day, specifically 1 μg/kg/day to 10 mg/kg/day, more specifically 10 μg/kg/day to 1 mg/kg/day, more specifically 50 μg/kg/day to 100 μg/kg/day. In one embodiment of the present invention, the pharmaceutical composition may be administered, but not limited to, 1 to 3 times a day.

In one embodiment of the present invention, a skin external composition for improvement or prevention of skin inflammation is provided. The skin external composition may contain an active ingredient that is a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides.

In another embodiment of the present invention, a cosmetic composition for improvement or prevention of skin inflammation is provided. The cosmetic composition may contain an active ingredient that is a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides.

In one embodiment of the present invention, external application composition or cosmetic composition may be provided in all forms appropriate for topical applications. For example, forms can be provided as solutions, emulsions obtained by dispersion of oil phase in water, emulsion obtained by dispersion of water in oil phase, suspension, solid, gel, powder, paste, foam or aerosol. These forms can be manufactured by conventional methods of the industry in the art.

In one embodiment of the present invention, the cosmetic composition may include, within levels that will not harm the main effect, other ingredients that can desirably increase the main effect. In one embodiment of the present invention, the cosmetic composition may additionally include moisturizer, emollient agents, surfactants, UV absorbers, preservatives, fungicides, antioxidants, pH adjusting agent, organic or inorganic pigments, aromatics, cooling agent or antiperspirant. The formulation ratio of the above-mentioned ingredients can be decided by those skilled in the art within levels that will not harm the purpose and the effects of the present invention, and the formulation ratio based on total weight of the cosmetic composition can be 0.01 to 5% by weight, specifically 0.01 to 3% by weight.

In one embodiment of the present invention, a food composition for inflammation prevention or suppression is provided. The food composition may contain with an active ingredient that is a peptide comprising of an amino acid sequence from SEQ 10 NO: 1 to SEQ ID NO: 161, a peptide comprising of amino acid sequence above 80% homology with above-mentioned sequences, or peptide fragment of above-mentioned peptides.

In one embodiment of the present invention, food composition is not limited to forms, but for example may be granules, powder, liquid, and solid forms. Each form can be formed with ingredients commonly used in the industry appropriately chosen by those skilled in the art, in addition to the active ingredient, and can increase the effect with other ingredients.

Decision for dosage on the above-mentioned active ingredient is within the level of those skilled in the art, and daily dosage for example may be 1 μg/kg/day to 10 mg/kg/day, more specifically 10 μg/kg/day to 1 mg/kg/day, more specifically 50 μg/kg/day to 100 μg/kg/day, but not limited to these numbers and can vary according to age, health status, complications and other various factors.

In one embodiment of the present invention, a use of prevention or treatment of inflammatory disease with a peptide comprising of an amino acid sequence from SEQ ID NO: 1 to SEQ ID NO: 161, a peptide comprising of amino acid sequence above 80% homology with above-mentioned sequences, or peptide fragment of above-mentioned peptides, is provided.

In one embodiment of the present invention, the method of prevention or treatment of inflammatory disease with applying peptides mentioned above in patients is provided.

In one embodiment of the present invention, a kit for prophylaxis or treatment of inflammatory diseases is provided. The kit may contain: a peptide with anti-inflammatory activity or a composition comprising of the peptide, wherein the peptide comprises any one amino acid sequence of SEQ ID NO: 1 to SEQ ID NO: 161, the peptide has above 80% homology with above-mentioned sequences, or the peptide is a fragment of the above-mentioned peptides; and instructions including at least one of administration dose, administration route, administration frequency, and indication of the peptide or composition.

The terms used herein is intended to be used to describe the embodiments, not to limit the present invention. Terms without numbers in front are not to limit the quantity but to show that there may be more than one thing of the term used. The term “including”, “having”, “consisting”, and “comprising” shall be interpreted openly (i.e. “including but not limited to”).

Mention of range of numbers is used instead of stating separate numbers within the range, so unless it is explicitly stated, each number can be read as separate numbers integrated herein. The end values of all ranges are included in the range and can be combined independently.

Unless otherwise noted or clearly contradicting in context, all methods mentioned herein can be performed in the proper order. The use of any one embodiment and all embodiment, or exemplary language (e.g., that use “like ˜”), unless included in the claims, is used to more clearly describe the present invention, not to limit the scope of the present invention. Any language herein outside of the claims should not be interpreted as a necessity of the present invention. Unless defined otherwise, technical and scientific terms used herein have meaning normally understood by a person skilled in the art that the present invention belongs to.

The preferred embodiments of the present invention are the best mode known to the inventors to perform the present invention. It may become clear to those skilled in the art after reading the statements ahead of the variations in the preferred embodiments. The present inventors hope that those skilled in the art can use the variations adequately and present invention be conducted in other ways than listed herein. Thus, the present invention, as allowed by the patent law, includes equivalents, and variations thereof, of the key points of the invention stated in the appended claims. In addition, all possible variations within any combination of the above-mentioned components are included in the present invention, unless explicitly stated otherwise or contradicting in context. Although the present invention is described and shown by exemplary embodiments, those skilled in the art will understand well that there can be various changes in the form and details without departing from the spirit of the invention and range, defined by the claims below.

Tumor necrosis factor (TNF), particularly TNF-α, is known to be released from inflammatory cells and cause various cytotoxic reactions, immunological reactions and inflammatory reactions. TNF-α is known to be involved in the occurrence and prolongation of many inflammatory and autoimmune diseases and further cause serious septicemia and septic shock when it is released into the blood and acts systemically, Because TNF-α is a factor associated widely with the immune system of a living body, the development of agents inhibiting TNF-α is actively carried out. TNF-α is biosynthesized in an inactive form and becomes an active form by being cleaved by protease; the enzyme responsible for the activation is called a tumor necrosis factor-converting enzyme (TACE). Thus, a substance inhibiting this TACE can treat, improve, or prevent diseases, pathologic conditions, abnormal conditions, troubles, adverse symptoms and the like ascribed to TNF-α.

High-mobility group box 1(HMGB1) protein exists in high concentrations in thymus, lymph nodes, testes, and in fetal liver, and with exception to liver and brain cells, usually exists inside of the nucleus. The said HMGB1 protein has 3 domains consisting of A-box, B-box, and C-terminal.

It was reported by Tracey et al., 1999 that HMGB1 protein has a role as a cytokine which induces inflammation, and the mechanism of said HMGB1's inflammation induction is by an external stimulus causing acetylation of HMGB1 which then moves from the nucleus into the cytoplasm. Afterward, it is known to be secreted out of the cell, or secreted out from the cell in necrosis. (Bonaldi T et at, EMBO J, (22)5551-60, 2003).

The invention is further described by the figures, the following examples and experiments, which are solely for the purpose of illustrating specific embodiments of this invention, and are not to be construed as limiting the scope of the invention in any way.

### EXAMPLE 1

### Synthesis of PEP-1 and Measurement of Anti-Inflamatory Activities of PEP-1 (SEQ ID NO: 1)

### Experiment 1. Synthesis of PEP-1 (SEQ ID NO: 1)

A peptide comprised of 16 amino acids with the chemical structure 1 as below having the sequence SEQ ID: 1 (PEP-1) derived from human telomerase was synthesized:

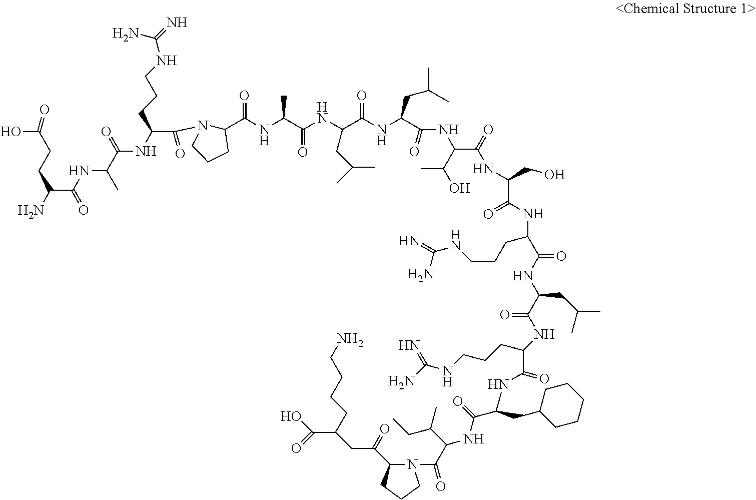

SEQ ID NO: 1 (PEP-1) was synthesized according to the existing method of solid phase peptide synthesis. In detail, the peptides were synthesized by coupling each amino acid from C-terminus through Fmoc solid phase peptide synthesis, SPPS, using ASP48S (Peptron, Inc., Daejeon ROK). Those peptides with their first amino acid at the C-terminus being attached to resin were used as follows:
NH₂-Lys(Boc)-2-chloro-Trityl Resin
NH₂-Ala-2-chloro-Trityl Resin
NH₂-Arg(Pbf)-2-chloro-Trityl Resin

All the amino acid materials to synthesize the peptide were protected by Fmoc at the N-terminus, and the amino acid residues were protected by Trt, Boc, t-Bu (t-butylester), Pbf (2,2,4,6,7-pentamethyl dihydro-benzofuran-5-sulfonyl) that can be dissolved in acid. Such as:

Fmoc-Ala-OH, Fmoc-Arg(Pbf)-OH, Fmoc-Glu(OtBu)-OH, Fmoc-Pro-OH, Fmoc-Leu-OH, Fmoc-Ile-OH, Fmoc-Phe-OH, Fmoc-Ser(tBu)-OH, Fmoc-Thr(tBu)-OH, Fmoc-Lys(Boc)-OH, Fmoc-Gln(Trt)-OH, Fmoc-Trp(Boc)-OH, Fmoc-Met-OH, Fmoc-Asn(Trt)-OH, Fmoc-Tyr(tBu)-OH, Fmoc-Ahx-OH, Trt-Mercaptoacetic acid.

HBTU[2-(1H-Berizotriazole-1-yl)-1,1,3,3-tetamethylaminium hexafluorophosphate]/HOSt [N-Hydroxybenzotriazole]/NMM[4-Methylmorpholine] were used as the coupling reagents. In 20% of DMF, piperidine was used to remove Fmoc. In order to remove the protection from residue or to separate the synthesized peptide from Resin, cleavage cocktail [TFA (trifluoroacetic acid)/TIS (triisopropylsilane)/EDT (ethanedithiol)/H₂O=92.5/2.5/2.5/2.5] was used.

Synthesized the peptide by using the solid phase scaffold combined to starting amino acid with the amino acid protection, reacting the corresponding amino acids separately, washing with solvent and deprotected, and repeating the process, After cutting off the synthesized peptide from the resin, it was purified by HPLC and verify for synthesis by MS, and then freeze-dried.

Specific synthesis process of PEP 1 is described by the following.
1) Coupling

Melted the amino acid (8 equivalent) protected with NH₂-Lys(Boc)-2-chloro-Trityl Resin, and coupling agent HBTU(8 equlv.)/HOBt(8 equiv.)/NMM(16 equiv.) and added to DMF, then let react in room temperature for 2 hours, then washed with DMF, MeOH, and DMF in that order,
2) Fmoc deprotection

Added 20% piperidine in DMF and reacted in room temperature for 5 minutes 2 times, then washed with DMF, MeOH, and DMF in that order.
3) Make the basic framework of peptide by repeating reactions 1 and 2 repeatedly,
4) Cleavage: Add Cleavage Cocktail to the completely synthesized peptide and separated the peptide from the resin.
5) Add cooling diethyl ether into obtained mixture, and then centrifugation is used to precipitate gathered peptide,
6) After purification by Prep-HPLC, check the molecular weight by LC/MS and lyophilize to produce in powder form.

### Experiment 2: Anti-Inflammatory Activity Measurement of PEP 1

Cell Lines Culture

Raw 264.7 macrophage cell (KCBL, 40071) from Korea Cell Bank was maintained in Dulbecco's modified Eagle's medium (DMEM; PAA, Austria) containing 10% fetal bovine serum (FBS; Gibco Laboratories), 100 unit/mL of streptomycin, and penicillin (Gibco Laboratories) at 37° C. with 5% CO₂. Raw264.7 cells were seeded into a 96-well plate at a density of 1×10⁶ cells/mL and incubated overnight.

On the following day, the medium was replaced with fresh medium and 5 μg/mL of peptide (obtained as described in Experiment example 1) was added to the cells. After 30 min incubation of cells with the peptide 50 μL of LPS (to a final concentration of 1 μg/mL) was added, and cells were incubated for additional 24 hr. The experimental sample with the induction of inflammatory response was treated with 1 μg/mL mL lipopolysaccharide (LPS; Sigma, USA) and control sample was treated with phosphate buffered saline (PBS; pH 7.2). The supernatant samples from each condition was collected in eppendorf tubes and subjected to further analysis.

### Experiment 2-1. NO Level Analysis

The level of nitric oxide (NO) was measured in Raw 264.7 cell (1×10⁶ cell/mi) using Griess reagent system (Promega, USA). Culture medium of 50 μl was added to a 96-well plate and Griess reagent I (NED) solution and Griess reagent II (Sulfanilamide solution) are added in the same amount. After 10 min incubation of cells with the reagents, the optical density at 540 nm was measured within 30 min using a microplate reader (Molecular Devices, USA). The concentration of NO was calculated by using a standard curve (0˜100 μM) of sodium nitrite.

As shown in Table 3 below, stimulation of cells with LPS increased the expression of NO, but in co-treatment with LPS and Pep1, the expression level of NO mentioned above decreased. NO is produced during inflammation, and the result showing Pep1 reduced NO level to 65% of the control strongly support the anti-inflammatory effect of Pep1.

TABLE 3
The measurement of anti-inflammatory effect
of human telomerase derived PEP 1

| | | NO Expression | Decreased NO |
|---|---|---|---|
| | | Level of | Expression |
| Test sample | | control (%) | Level (%) |
| PBS | | 0 | — |
| LPS 1 μg/mL | PBS | 100 | 0 |
| | PEP 1 (0.5 μg/mL) | 35 | 65 |

### Experiment 2-2. Analysis of Cytokine Inhibitory Effect

To investigate the effect of PEP1 on inhibiting pro-inflammatory cytokine production RAW 2647 cell were pre-treated with PEP 1 at a concentration of 5 μg/mL challenged with LPS at a concentration of 1 μg/mL, and cells were further incubated for 24 hr. The supernatant samples containing cell culture medium was collected and analyzed for the cytokine levels using ELISA kits (eBioscience, San Diego).

96 wells plates were coated with 100 μl of capture antibodies (diluted in coating buffer to the concentration recommended by manufacturer's protocol) overnight at 4° C. Then, after washing the plates 5 times, 200 μl of assay diluents was added to each well and incubated for 1 hr at room temperature for blocking. After washing each well with wash buffer five times, cell culture sample or each cytokine standard protein sample was diluted and 100 μl of each added into each well. The plate containing samples were incubated overnight at 4° C.

Then, after washing the plate five times with the wash buffer, 100 μl of secondary antibody conjugated to avidin was added and incubated for 1 hr at room temperature.

Following incubation with the secondary antibody, the plate was washed five times and incubated with 100 μl of avidin-HRP (BD Bioscience) for 30 min at room temperature. After washing the plate seven times, 100 μl of TMB solution (Pierce) was added and incubated for 15 min at room temperature. The reaction was stopped by adding 50 μl of 2N H₂SO₄ in each well The optical density at 450 nm was measured using a microplate reader. Statistical analysis was performed by variance analysis using ANOVA procedure of SPSS program, and verified the significance between analyses using Duncan's multiple range test.

### Experiment 2-3. IL-6 Secretion Measurement

As shown in Table 4 below, treatment with LPS alone increased the cytokine IL-6 (interleukin-6) secretion. However, co-treatment with LPS and PEP-1 showed a decrease in the level of the pro-inflammatory cytokine IL-6 secretion. More importantly, after the treatment with PEP-1, the level of pro-inflammatory cytokine secretion decreased by more than 70%, which indicates a robust anti-inflammatory effect of Pep1.

TABLE 4
Cytokine IL-6 production inhibition by PEP-1

cytokine IL-6 production

| Test sample | | % of control | inhibition % |
|---|---|---|---|
| PBS | | 0 | — |
| LPS 1 μg/ml | PBS | 100 | 0 |
| | PEP 1 (5 μg/ml) | 28 | 72 |

### Experiment 2-4. HMGB1, TNF-α, COX-2 Expression Inhibition

Protein expression level was determined by Western blot analysis. Cells grown in PEP-1 containing medium were washed with PBS, treated with 0.05% trypsin-EDTA, and collected by centrifugation. The collected cells were dissolved in an appropriate volume of lysis buffer. Intracellular debris was pelleted by centrifugation, and equal amount of protein from each sample was separated by SDS-polyacrylamide gel electrophoresis. The separated protein was transferred to nitrocellulose membrane (Schleicherand Schuell, Keene, NH, USA), then was tested for the antibody specific for each protein. The membrane was incubated with ECL (enhanced chemiluminoescence) solution(Amersham Life Science Corp., Arlington Heights, IL, USA), exposed to X-ray, and the level of protein expression was analyzed according to the exposure level shown on the X-ray film.

Western blot analysis was performed to determine the inhibitory effect of Ppep1 on the cytokine protein expression. As shown in Table 5 below, stimulation of cells with LPS increased the expression of cytokines; HMGB1, TNF-α and COX. However, if cells were treated with both LPS and Pep1, the expression level of pro-inflammatory cytokines mentioned above decreased. The result showing the treatment with Pep1 decreased pro-inflammatory cytokine levels by more than 70% provide strong evidence supporting the anti-inflammatory effect of Pep1.

TABLE 5
The measurement of inhibitory effect of Pep1 on
pro-inflammatory cytokine expression level.

| | | Cytokine Expression Level (band | | |
|---|---|---|---|---|
| | | intensity) % of control | | |
| Test sample | | HMGB1 | TNF-α | COX-2 |
| PBS | | — | — | — |
| LPS 1 μg/ml | PBS | 100 | 100 | 100 |
| | PEP 1 (5 μg/ml) | 30 | 25 | 22 |

### EXAMPLE 2

TNF-α Inhibitory Effect of PEP RIA Series (Sequence No. 1 to 161) Peptides

Based on the results from Example 1 in which the SEQ ID NO: 1 (PEP1) has the TNF-α inhibitory effect, experiment using peptides SEQ ID NO: 1 to 161 were carried out to confirm their TNF-α inhibitory effect. The synthesis of peptides SEQ ID NO: 1 to 161 used the same method mentioned above in Example 1 (method used for synthesis of PEP1), but the amino acids added were different.

### Experiment 1: Cell Culture

PBMC (peripheral blood mononuclear cells) layer was separated from blood samples (50 ml) collected in healthy subjects using Biocoll Separating Solution (Biochrom AG, Berlin, Germany). The collected PBMC were enriched in RPMI 1640 medium containing 20% human serum for 30 mins, and then transferred to 100-mm polystyeren cell culture plate coated with human serum for incubation for 2 hrs at 37° C., 5% CO² incubator. Monocytes were detached from the bottom of the plate using cold PBS, and incubated to reach the number of 1×10⁵ cell/well in 96-well plate with RPMI 1640 medium (supplemented with penicillin-streptomycin; 100 mg/ml, human serum; 20%) over night.

### Experiment 2: Analysis of TNF-α Inhibitory Effect in PBMC-Derived Monocyte

ELISA was performed to find out how the peptides of the PEP RIA series influence TNF-α level. PBMC-derived monocytes were incubated to reach the number of 1×10⁵ cells per well in a 96-well plate and then treated with LPS (lipopolysaccharide; 10 ng/ml, Sigma) for 2 hours. To the monocytes that were washed three times with PBS, OPTI-MEM culture medium was added to induce cell starvation for an hour, 4 μM of the peptide was taken out and incubated for 2 hours. There were three negative control groups. The first group was not treated with anything. The second group that was treated with estrogen (in this experiment, estradiol was used as a kind of estrogen). The third group was treated with LPS (10 ng/ml) or with LPS (10 ng/mi) as well as estrogen (20 nM). PEP1 that was confirmed to have TNF-α Inhibiting activity was used as a positive control to measure TNF-α inhibiting activity. After incubation, TNF-α was measured by following the ELISA kit manual (R&D, Minneapolis, MN, USA), The details of quantification method can be found in Experiment 2.2 of Example 1.

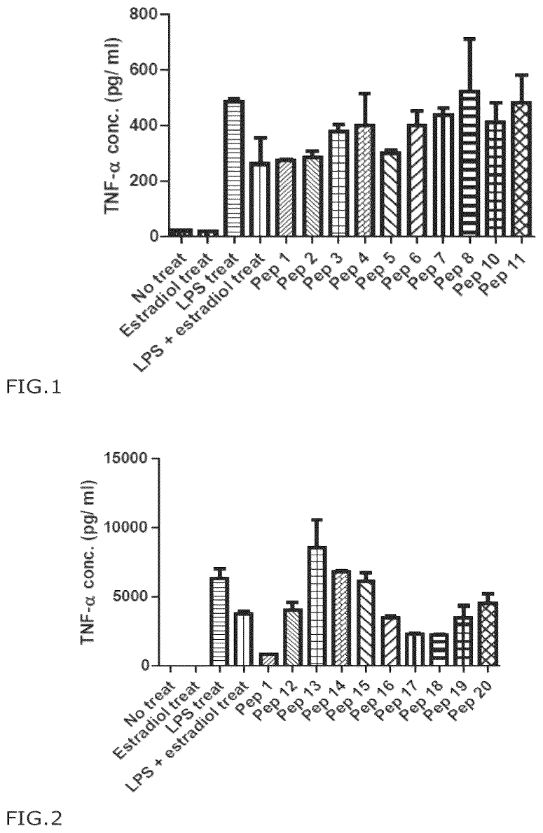

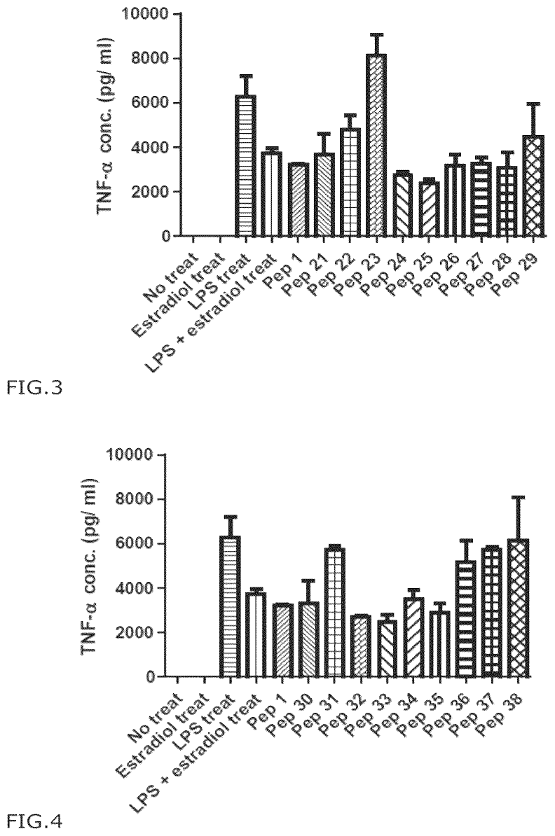

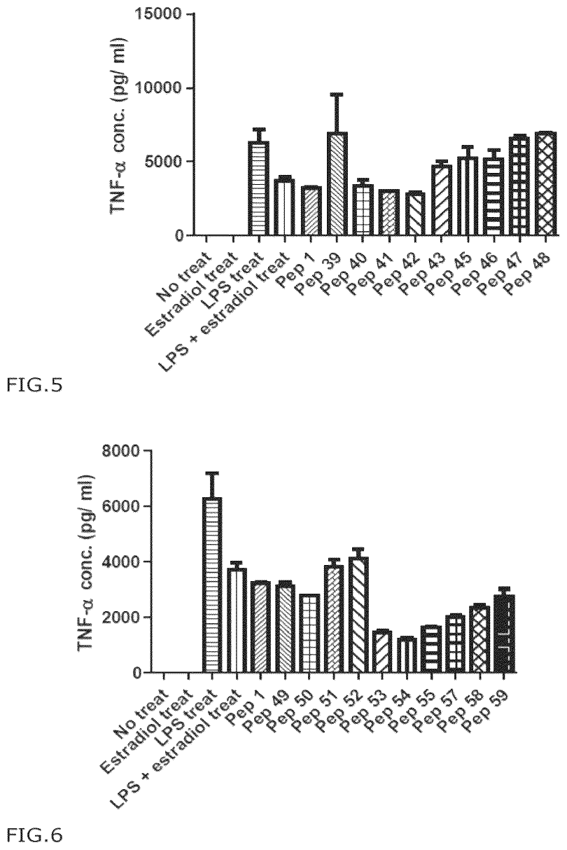

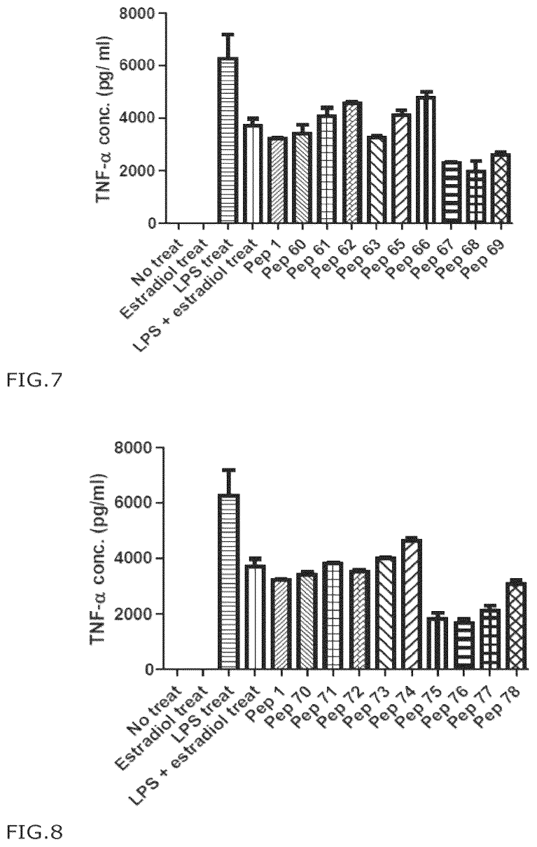

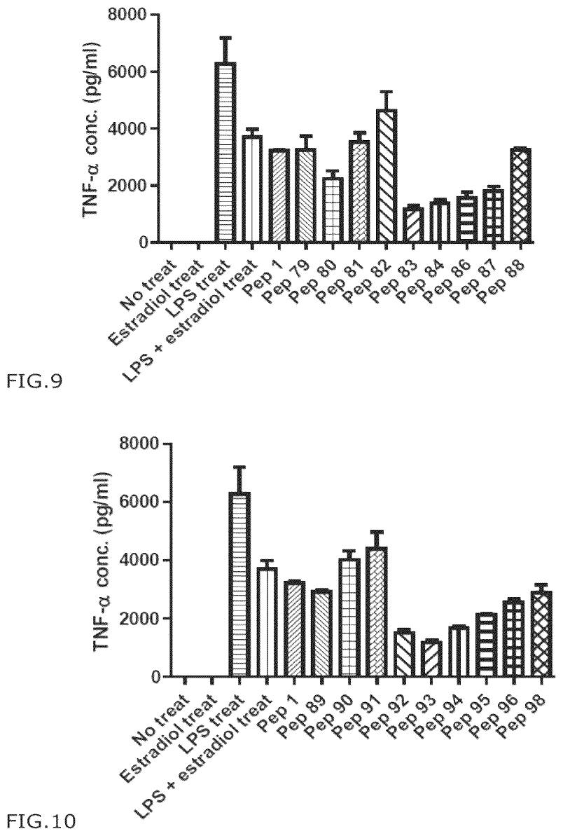

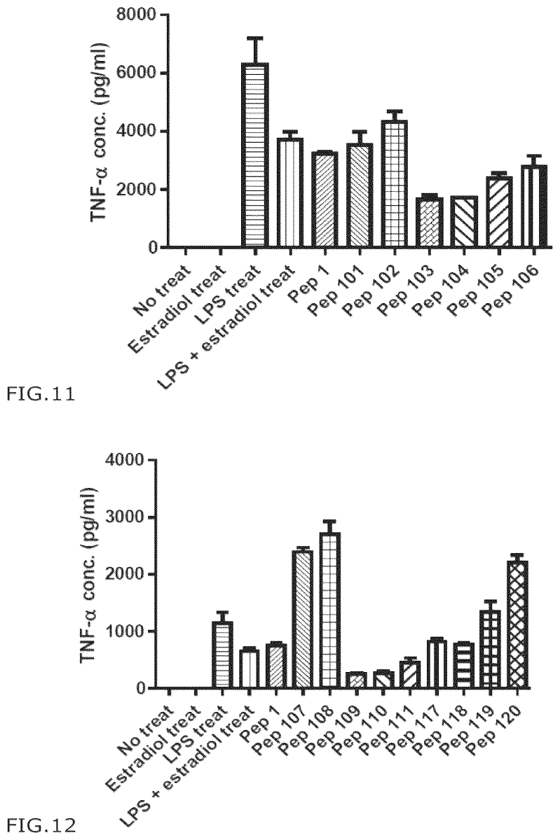

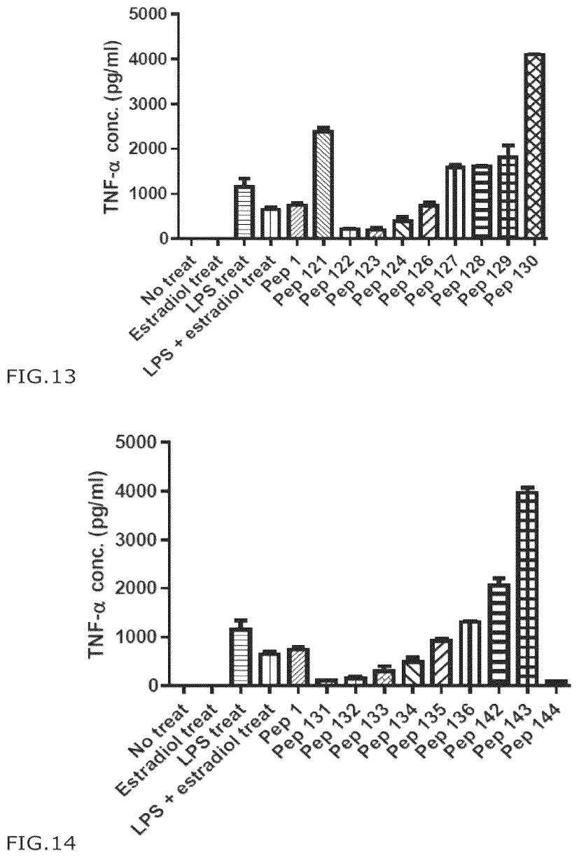

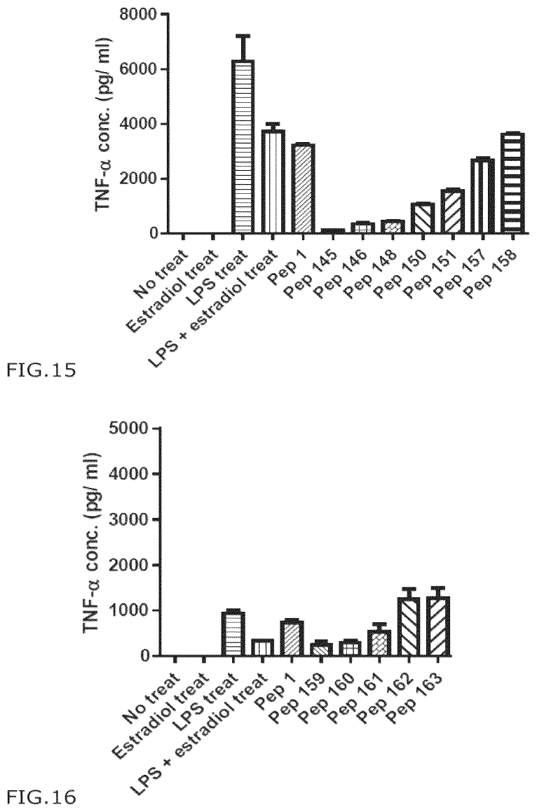

Using the method stated above, Peptides with TNF-α inhibiting effect were screened. PBMC-derived monocytes were stimulated with IPS (10 ng/ml), which is endotoxin, for 2 hours and were induced to starve by adding OPTI-MEM for 1 hour. After that, 4 μM of 161 peptides were treated and incubated for 2 hrs. The amount of TNF-α in the cell culture medium was measured using ELISA, and the peptides with TNF-α inhibiting effect were screened by comparing to the negative and positive controls (FIG. 1 to FIG. 16).

The followings are the peptides that showed TNF-α Inhibiting effect when compared to the control group that was treated with only LPS: SEQ ID NO: 1 to SEQ ID NO: 6, SEQ ID NO: 9, SEQ ID NO: 11, SEQ ID NO: 14 to 21, SEQ ID NO: 23 to SEQ ID NO: 37, SEQ ID NO: 39 to SEQ ID NO: 44, SEQ ID NO: 47 to SEQ ID NO: 53, SEQ 10 NO: 55 to SEQ ID NO: 61, SEQ ID NO: 63 to SEQ ID NO: 82, SEQ ID NO: 84 to SEQ ID NO: 94, SEQ ID NO: 96, SEQ ID NO: 99 to SEQ ID NO: 104, SEQ ID NO: 107 to SEQ ID NO: 109, SEQ ID NO: 115, SEQ ID NO: 116, SEQ ID NO: 120 to SEQ ID NO: 122, SEQ ID NO: 124, SEQ ID NO: 129 to SEQ ID NO: 133, SEQ ID NO: 142 to SEQ ID NO: 144, SEQ ID NO: 146, SEQ ID NO: 148, SEQ ID NO: 149, and SEQ ID NO: 155 to SEQ ID NO: 159.

Also, the followings are the peptides that showed TNF-α inhibiting effect when compared to the group treated with LPS and estrogen: SEQ ID NO: 15 to SEQ ID NO: 18, SEQ ID NO: 23 to SEQ ID NO: 27, SEQ 10 NO: 29, SEQ ID NO: 31 to SEQ ID NO: 34, SEQ ID NO: 39 to SEQ ID NO: 41, SEQ ID NO: 47, SEQ ID NO:48, SEQ ID NO: 51 to SEQ ID NO: 53, SEQ ID NO: 55 to SEQ ID NO: 58, SEQ 10 NO: 61, SEQ ID NO: 65 to SEQ ID NO: 68, SEQ ID NO: 70, SEQ ID NO: 73 to SEQ ID NO: 79, SEQ ID NO: 81, SEQ ID NO: 82, SEQ ID NO: 84 to SEQ ID NO: 87, SEQ ID NO: 8=90 to SEQ ID NO: 94, SEQ ID NO: 96, SEQ ID NO: 99, SEQ ID NO: 101 to SEQ ID NO: 104, SEQ ID NO: 107 to SEQ ID NO: 109, SEQ ID NO: 120, SEQ ID NO: 121, SEQ ID NO: 129 to SEQ ID NO: 132, SEQ ID NO: 142 to SEQ ID NO: 144, SEQ ID NO: 146, SEQ ID NO: 148, SEQ ID NO: 149, and SEQ ID NO: 157 to SEQ ID NO: 159.

### Experiment 3: Analysis of Peptides Affecting TNF-α Level in THP1 Cell Line

Experiment was carried out using THP-1 cell line (American Type Culture Co lection (ATCC), Manassas, VA, USA) which is human acute monocytic leukemia.

THP-1 cells were incubated to reach the number of 1×10⁵ cells per well in a 96-well plate with RPMI 1640 medium for 24 hrs, followed by addition or 100 μM of PMA (phorbol 12-myristate 13-acetate) for the differentiation into macrophage. After differentiation of THP-1 into macrophage by PMA for a day, LPS was treated for 2 hrs and washed off. Starvation for an hour and PEP1 treatment followed.

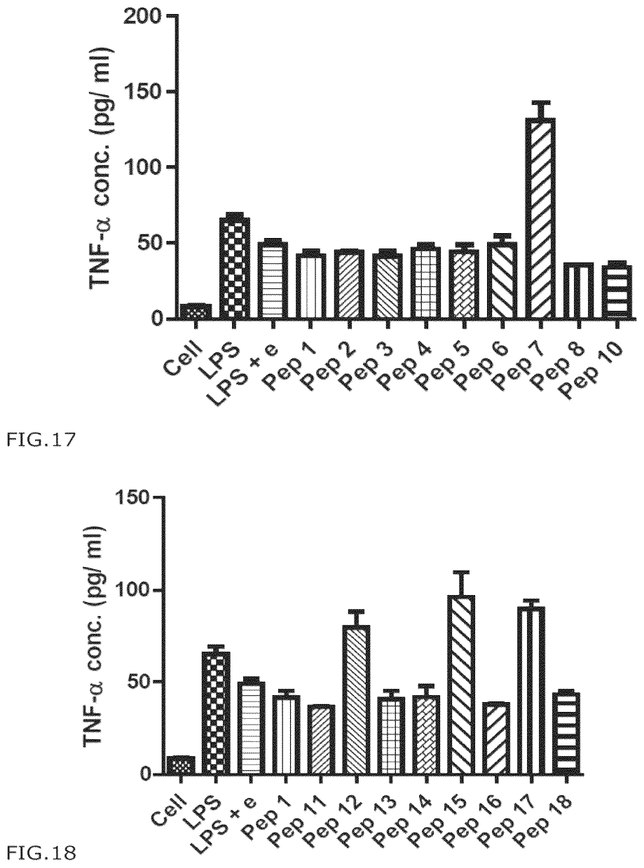

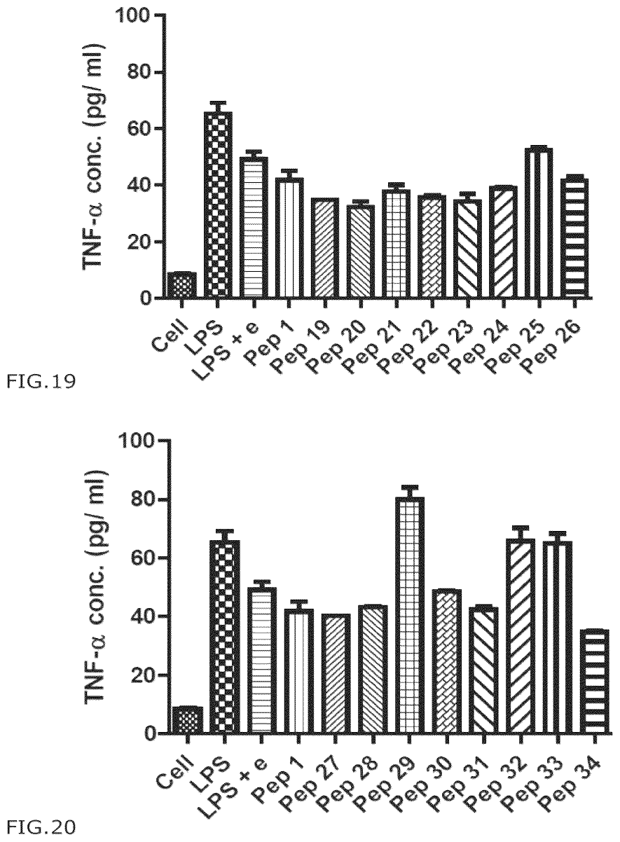

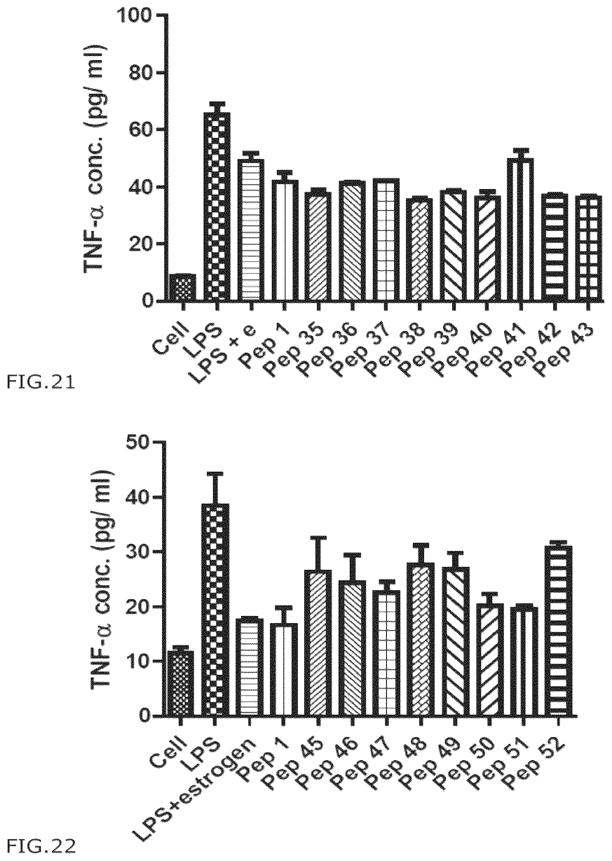

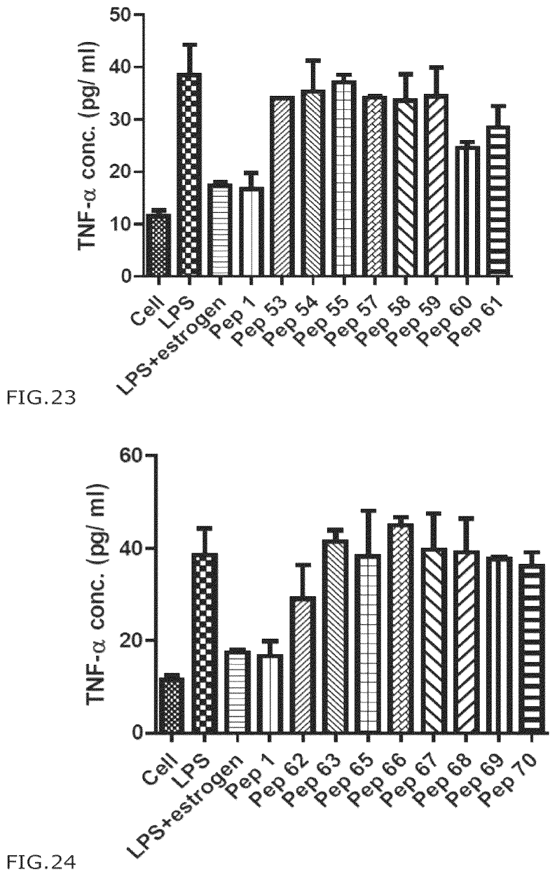

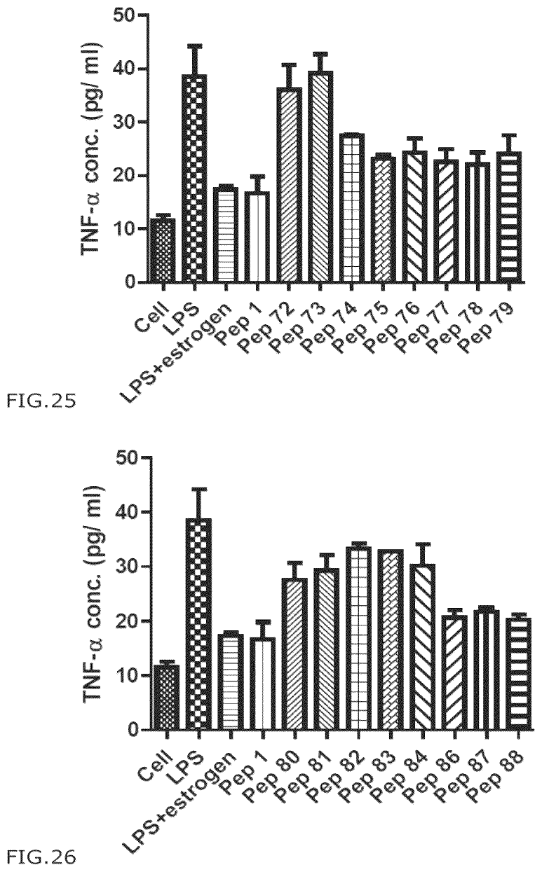

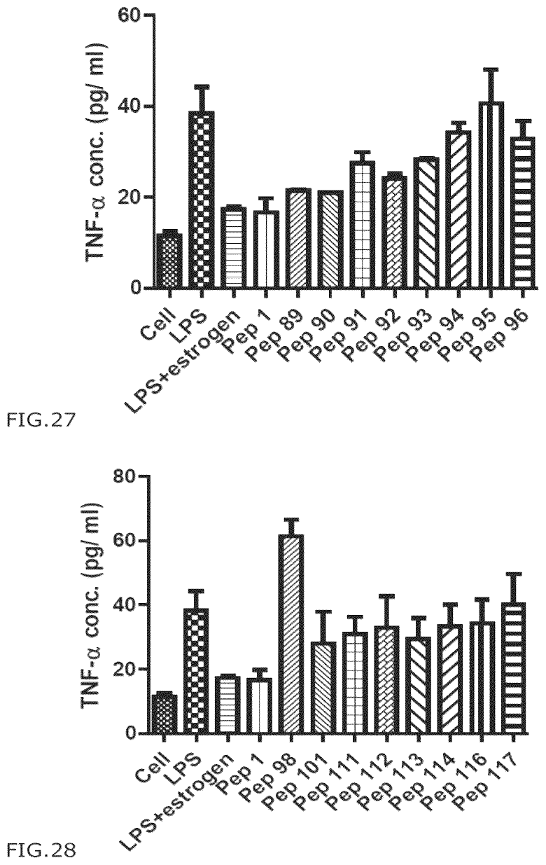

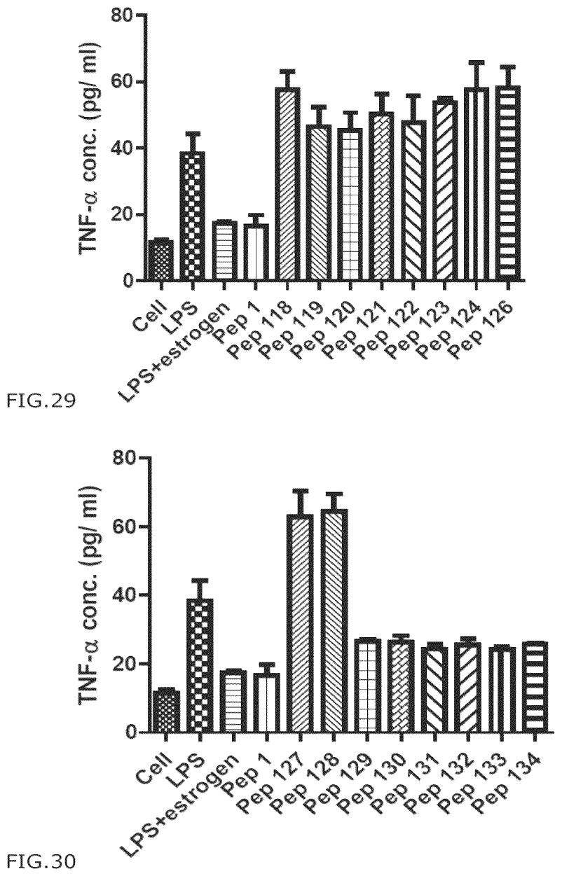

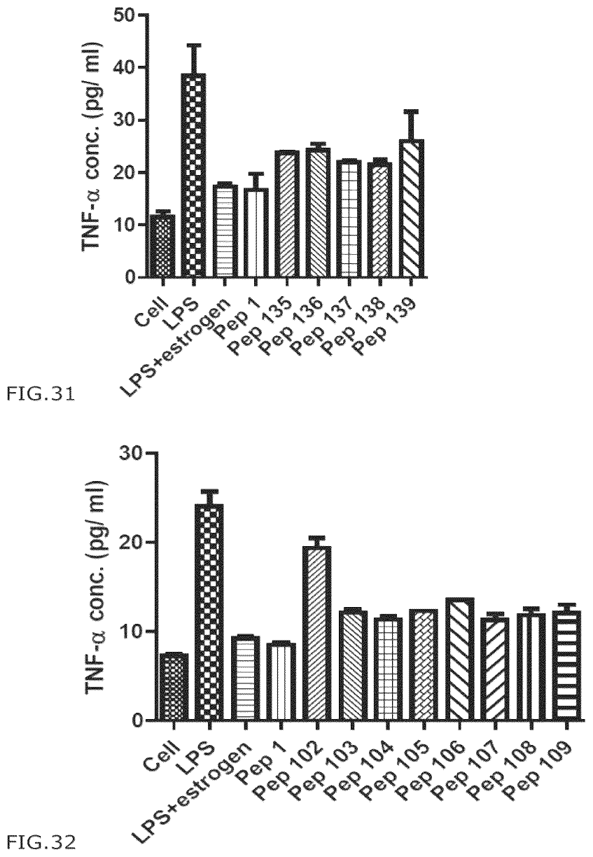

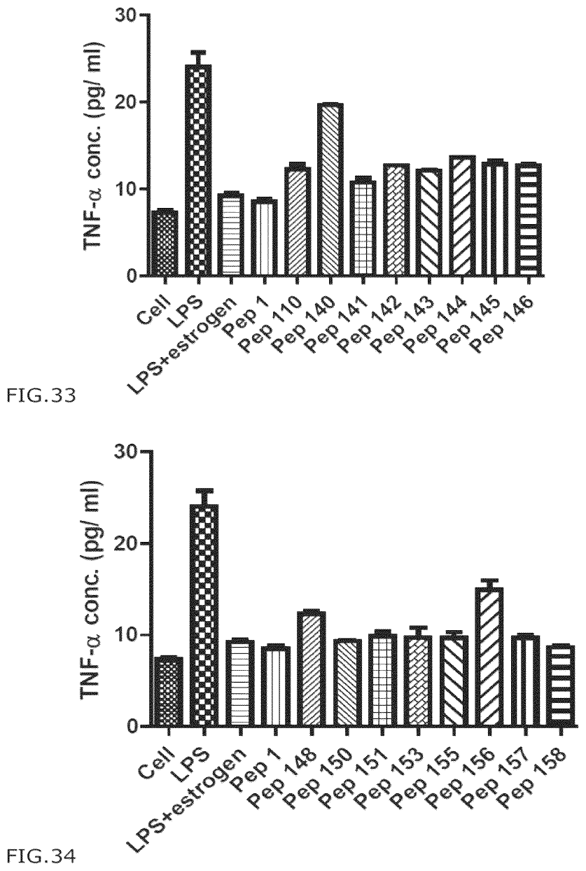

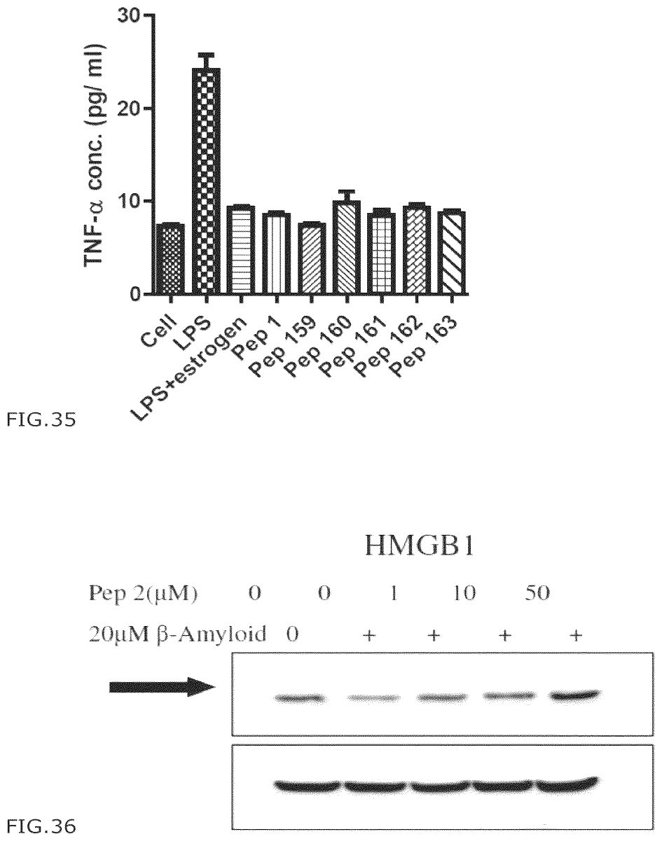

THP-1 cell differentiated by PMA was treated with LPS (lipopolysaccharide; 10 ng/ml, Sigma) for 2 hours, followed by 2 times washes with PBS. To the cells, OPTI-MEM culture medium was added to induce cell starvation for an hour, and 1 μM of 161 peptides was taken out and incubated for an hour. After incubation, TNF-α level was measured by using the ELISA kit and the peptides which reduce the TNF-α level were screened (FIG. 17 to FIG. 35).

As a result, peptide SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 27, SEQ ID NO: 29, SEQ ID NO: 30, SEQ ID NO: 32 to SEQ ID NO: 53, SEQ ID NO: 55 to SEQ ID NO: 60, SEQ ID NO: 67, SEQ ID NO: 68, SEQ ID NO: 70, SEQ ID NO: 72 to SEQ ID NO: 82, SEQ ID NO: 84 to SEQ ID NO: 92, SEQ ID NO: 94, SEQ ID NO: 99 to SEQ ID NO: 112, SEQ ID NO: 114, SEQ ID NO: 127 to SEQ ID NO: 144, SEQ ID NO: 146, SEQ ID NO: 148, SEQ ID NO: 149, SEQ ID NO: 151 and SEQ ID NO: 153 to SEQ ID NO: 161 appeared to reduce the TNF-α level compared to control group treated only with LPS.

In addition, SEQ ID NO: 1 to SEQ ID NO: 5, SEQ ID NO: 7, SEQ ID NO: 9, SEQ ID NO: 10, SEQ ID NO: 12, SEQ ID NO: 13, SEQ ID NO: 15, SEQ ID NO: 17 to SEQ ID NO: 2:3, SEQ ID NO: 25 to SEQ ID NO: 27, SEQ ID NO: 29, SEQ ID NO: 30, SEQ ID NO: 33 to SEQ ID NO: 43, SEQ ID NO: 156, SEQ ID NO: 157 and SEQ 10 NO: 159 were selected as peptides which reduce the expression level of TNF-α compared to that of group treated with LPS and estrogen.

### EXAMPLE 3

Effects of Peptides (SEQ ID No: 1 to 161) on Inflammation by Amyloid-β Protein

HMGB1 first undergoes acetylation and translocation to cytoplasm by external stimulation. Then it is secreted out of the cell, therefore serving the role of inflammation-causing cytokine. Because when one has an inflammation due to such activity, HMGB1 protein is secreted from the cell, and patients with inflammatory diseases such as Churg strauss syndrome, rheumatoid arthritis and Sjogren's syndrome will present with elevated serum levels of HMGB1, Hence, if nucleus contains large amount of HMGS1 even when there is a stimulus that causes inflammation, it is suggestive of the fact that HMGB1 is not being secreted out of the cell, which means inflammation is being suppressed.

### Experiment 1. Cell Culture

Undifferentiated PC12 cells (ATCC, Rockville, MD, USA) were maintained in logarithmic-phase growth on poly-l-lysine (Sigma, Saint Louis, MO, USA)—precoated 100 mm dishes (Corning, PA, USA) in RPMI 1640 medium (GIBCO, Grand Island, NY, USA) containing 10% heat-inactivated horse serum, 5% heat-inactivated fetal bovine serum, 100 units/ml penicillin, and 100 g/ml streptomycin, Cultures were incubated at 37° C. in a humidified atmosphere with 5% CO². The cultures were grown to 50% confluence and were harvested in Ca²⁺/Mg²⁺-free Hank's balanced salt solution containing 1 mM EDTA. Cells were plated at a density of 1×10⁶ cells/100 mm dish and incubated for 24 hrs. For neuronal differentiation, PC12 cells were serum-starved for 12 hrs (RPMI1640 medium containing 100 units/ml penicillin and 100 g/ml streptomycin without horse serum or fetal bovine serum); thereafter, the cells were maintained in serum-free medium, After two days the medium was replaced with fresh serum-free medium. On day three, NGF (50 ng/ml, Sigma, Saint Louis, MO, USA) was added to the medium, and the cultures were maintained for an additional three days. After differentiation, nPC12 cells were incubated with 20 μM amyloid-β with several concentrations of the peptides [0 (control), 1, 10, and 50 μM] for 48 hrs.

### Experiment 2. Western Blot Analysis

Levels of HMGB1 were analyzed by western blotting. Briefly, 5×10⁶ cells were washed twice in cold PBS, incubated for 10 min on ice in lysis buffer (50 mM Tris (pH 8.0), 150 mM NaCl, 0.02% sodium azide, 0.2% SDS, 100 μg/mi phenyl methyl sulfonyl fluoride (PMSF), 50 μl/mi aprotinin, 1% Igepal 630, 100 mM NaF, 0.5% sodium deoxy choate, 0.5 mM EDTA, 0.1 mM EGTA]; unbroken cells and nuclei were pelleted by centrifugation for 10 min at 2000×g and the lysates were cleared by centrifugation at 10,000×g. The antibodies used were: anti-HMGB1 (1:1000, Cell Signaling, Beverly, MA, USA) and anti-β-tubulin (1:1000, Cell Signaling, Beverly, MA, USA). The membranes were washed with Tris-buffered saline containing 0.05% Tween-20 (TBST), and then processed using HRP-conjugated anti-rabbit antibody (Amersham Pharmacia Biotech, Piscataway, NJ, USA) followed by ECL detection (Amersham Pharmacia Biotech,), The blots were quantified with an image analyzer (GE Healthcare, ImageQuant LAS 4000).

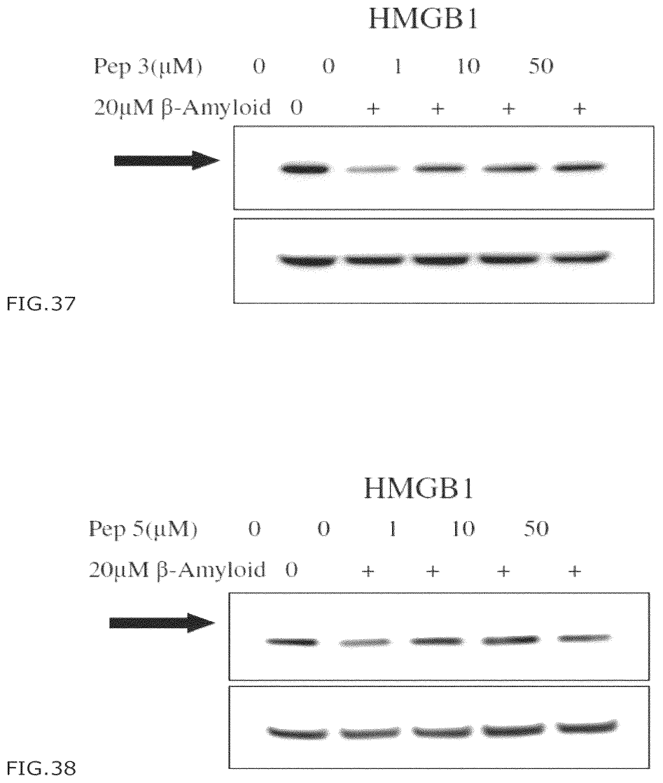

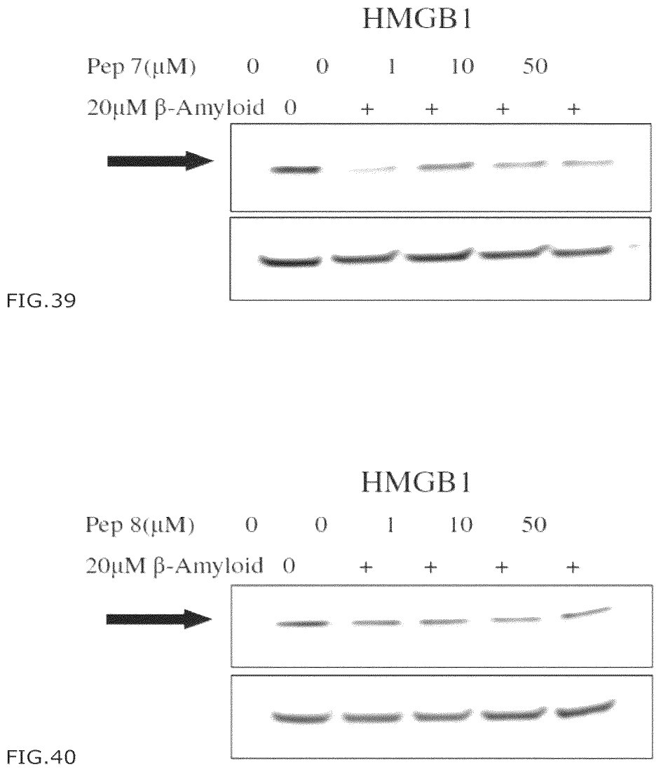

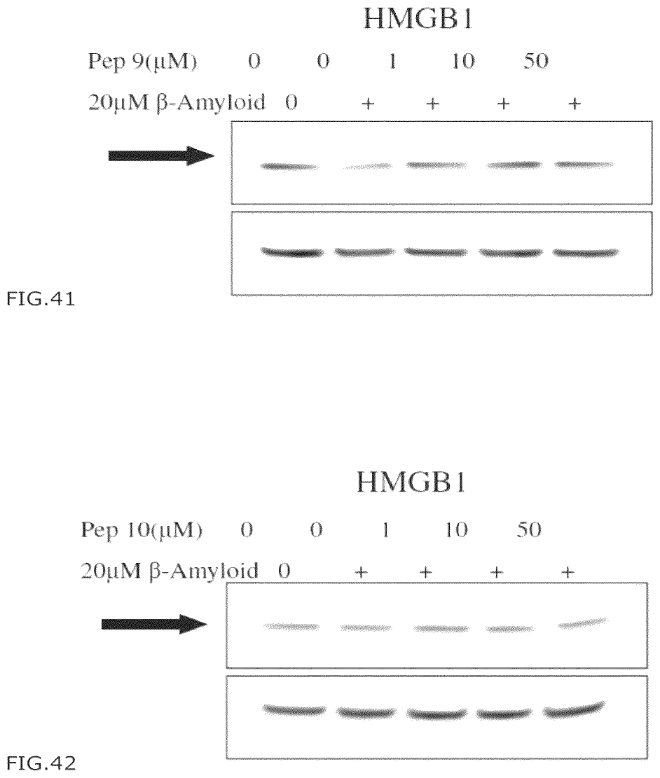

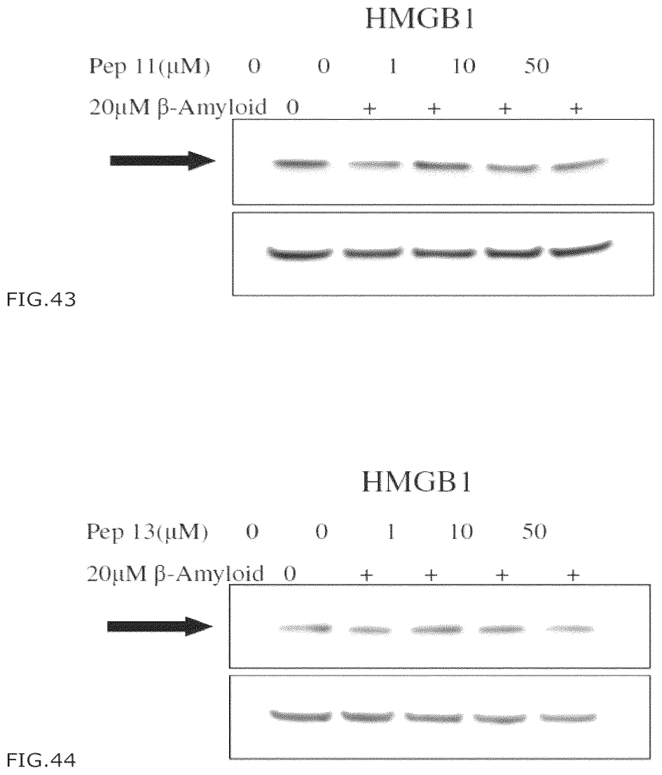

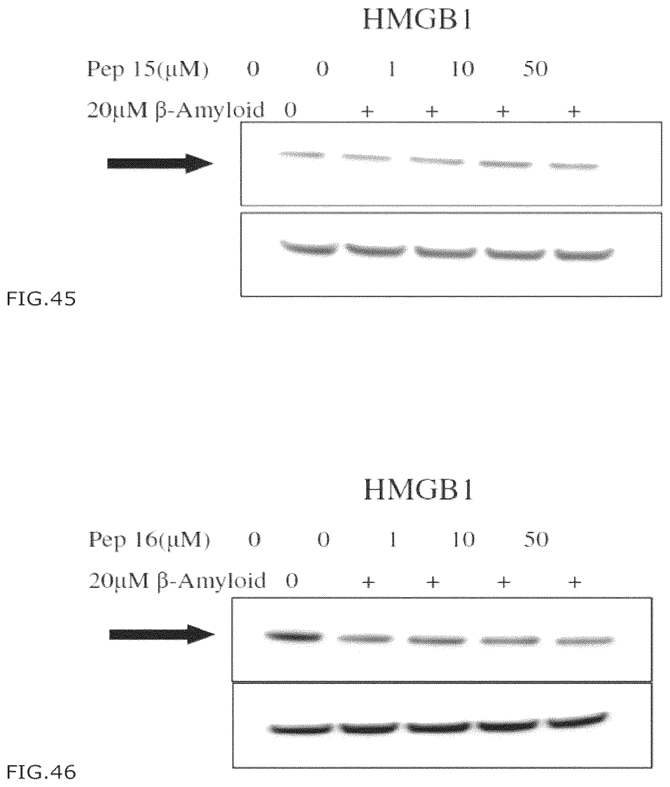

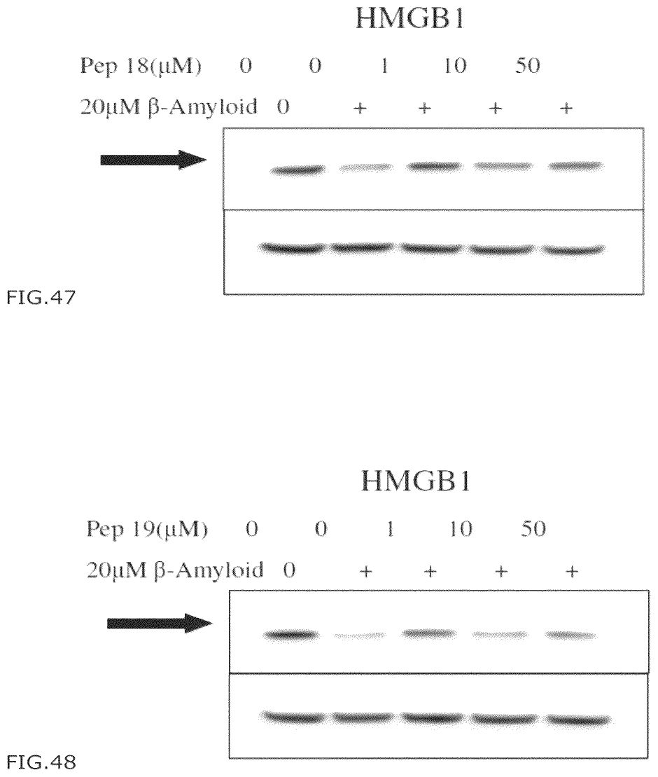

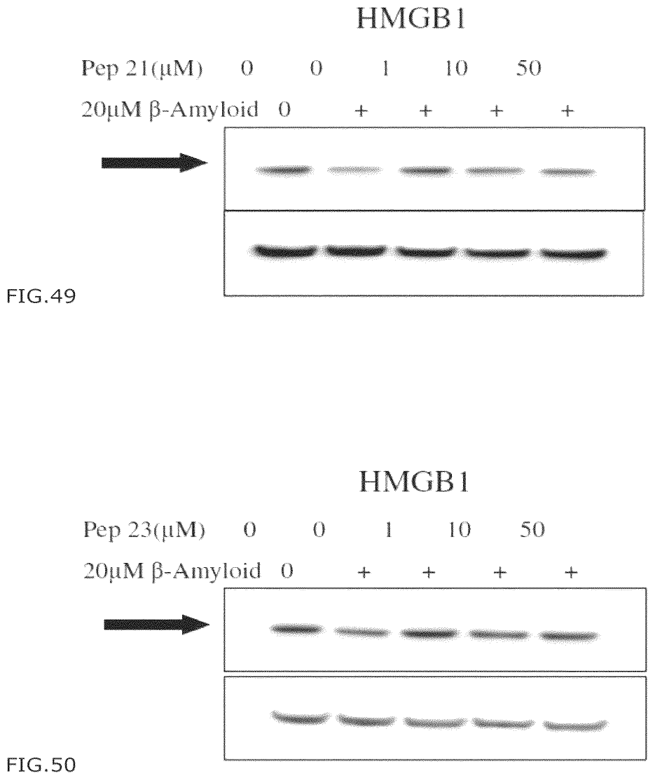

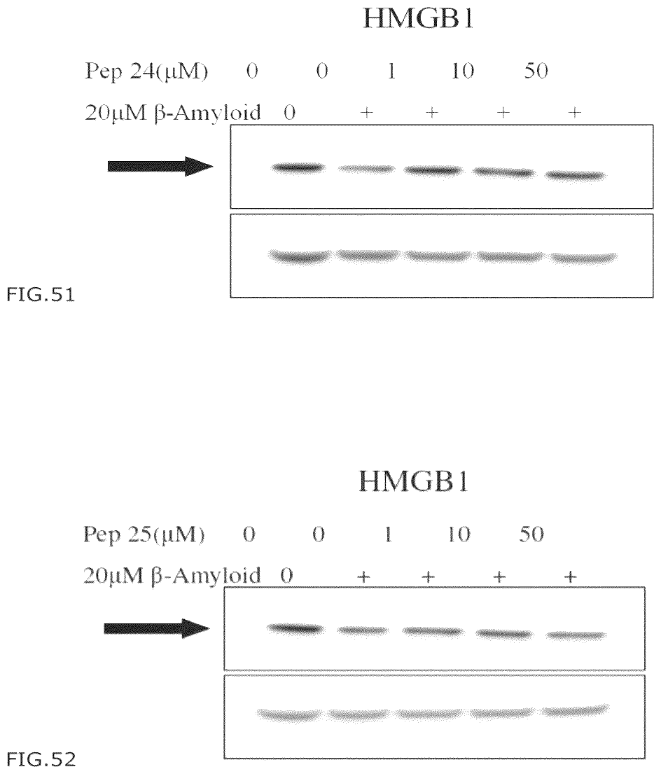

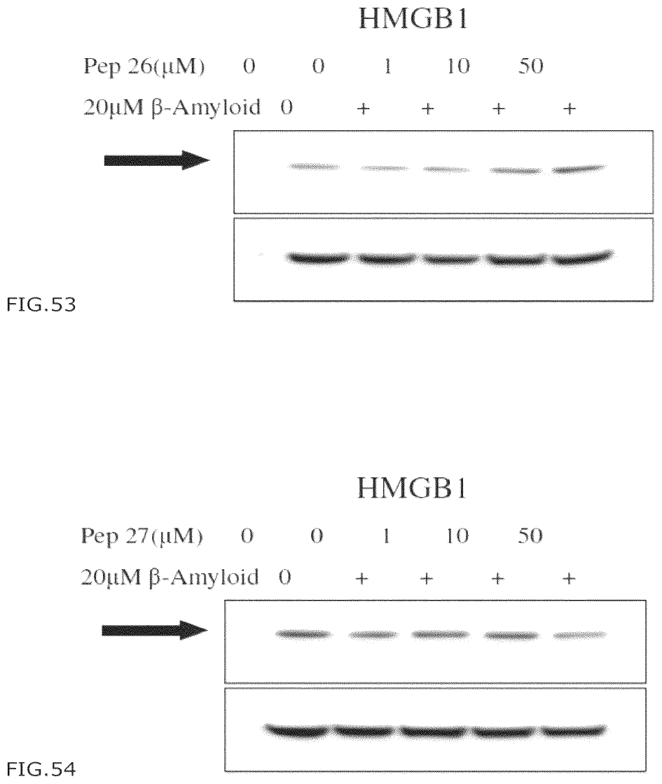

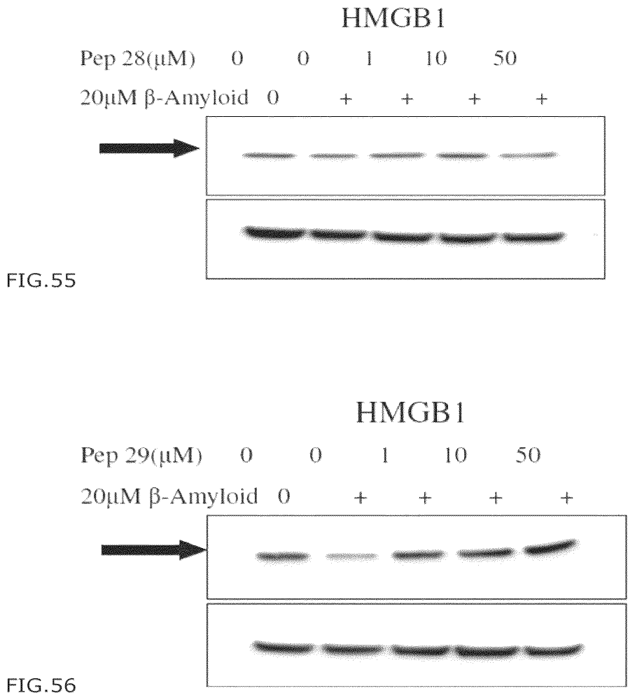

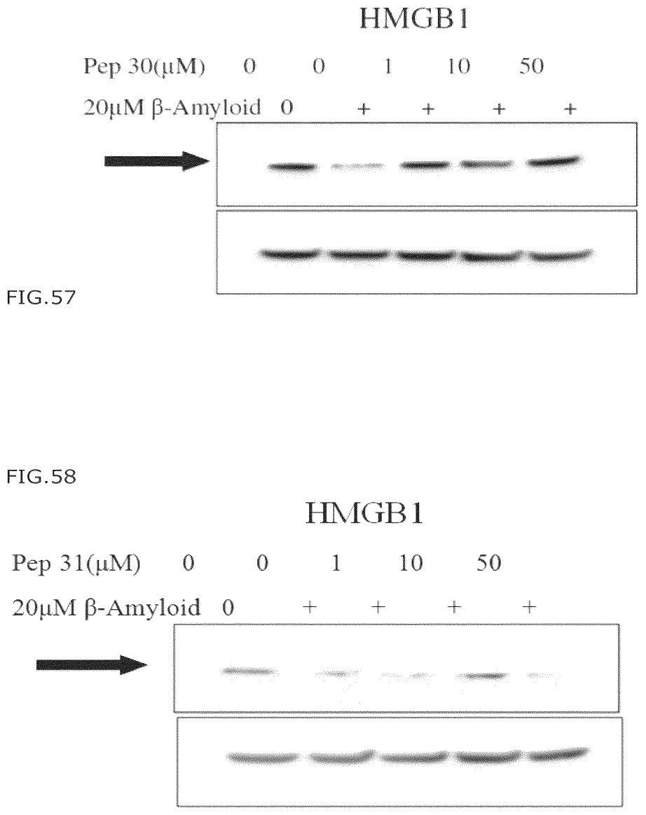

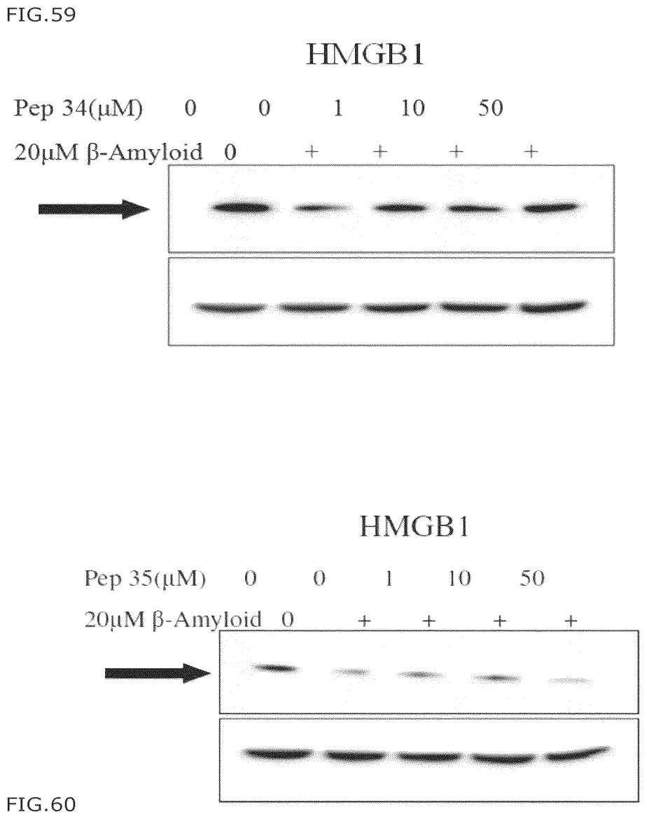

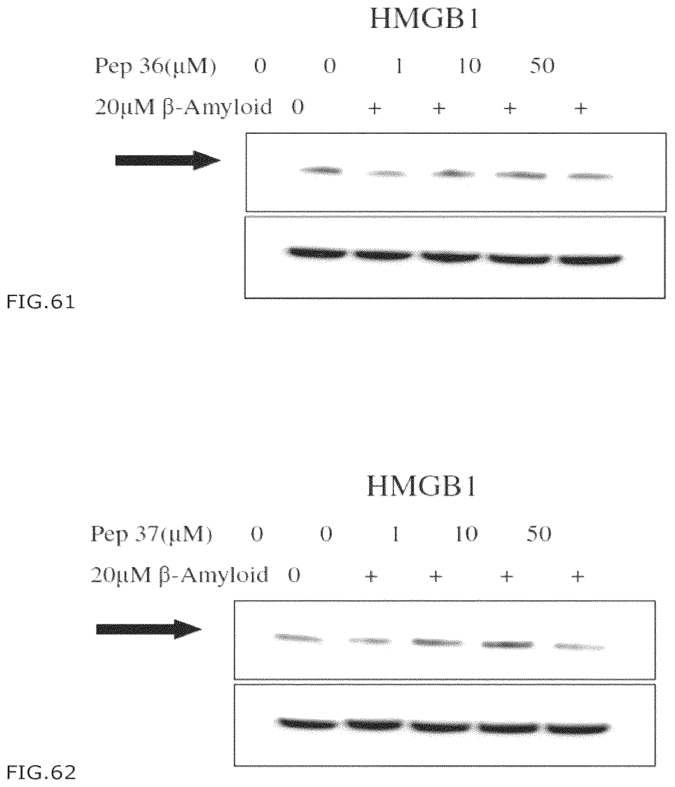

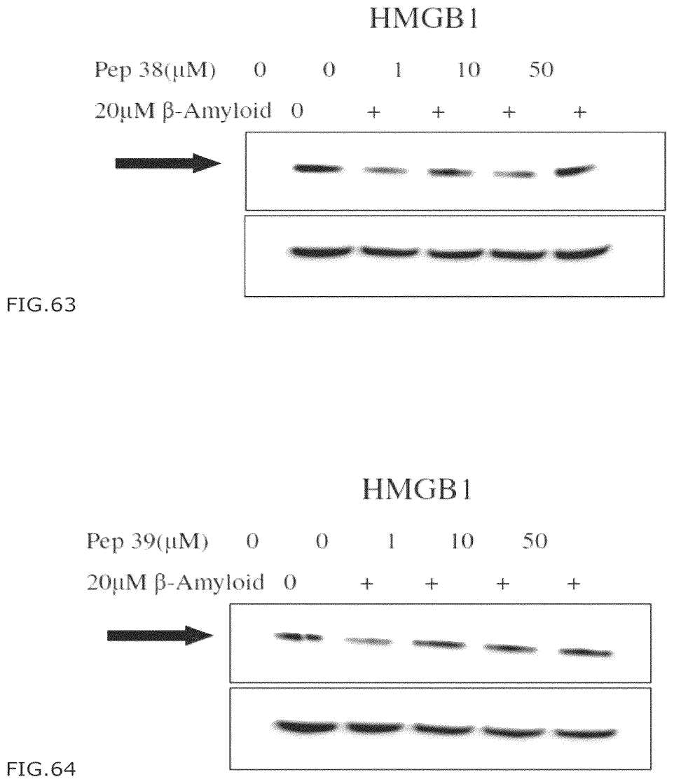

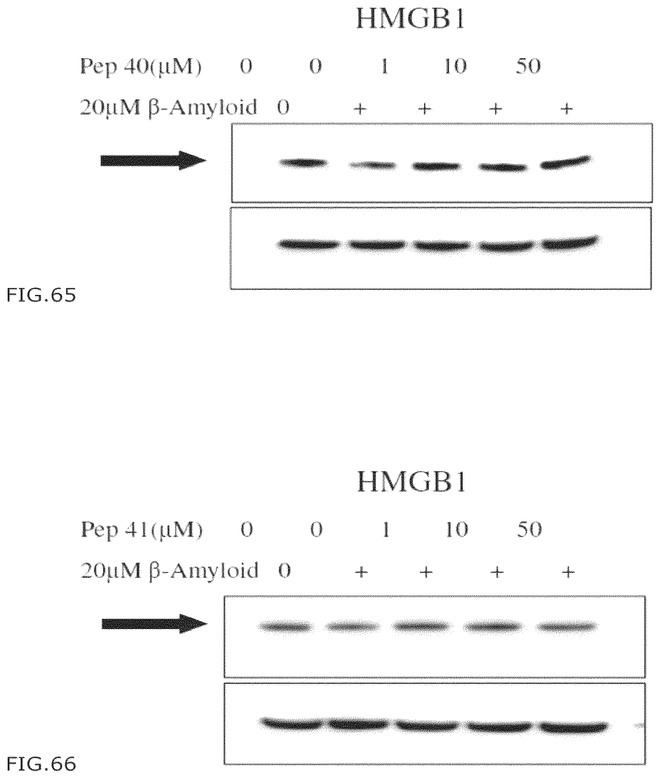

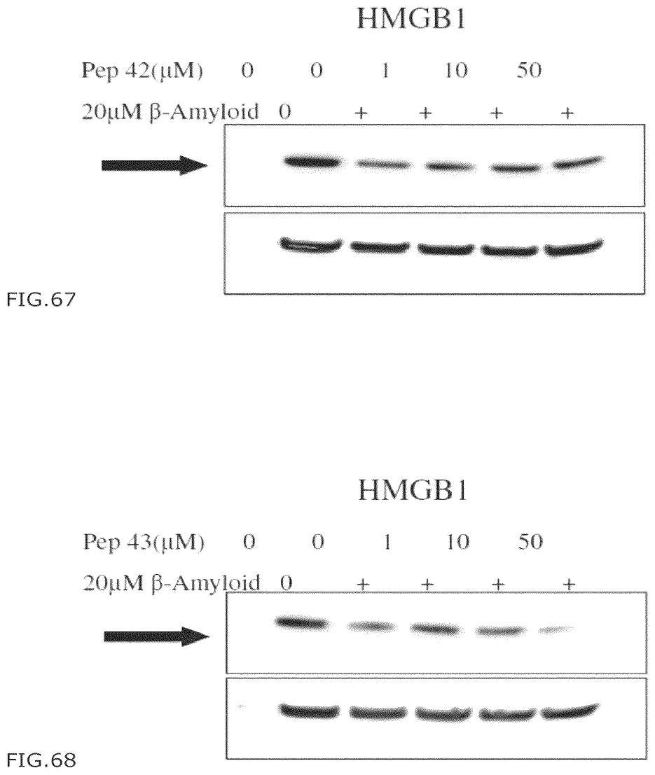

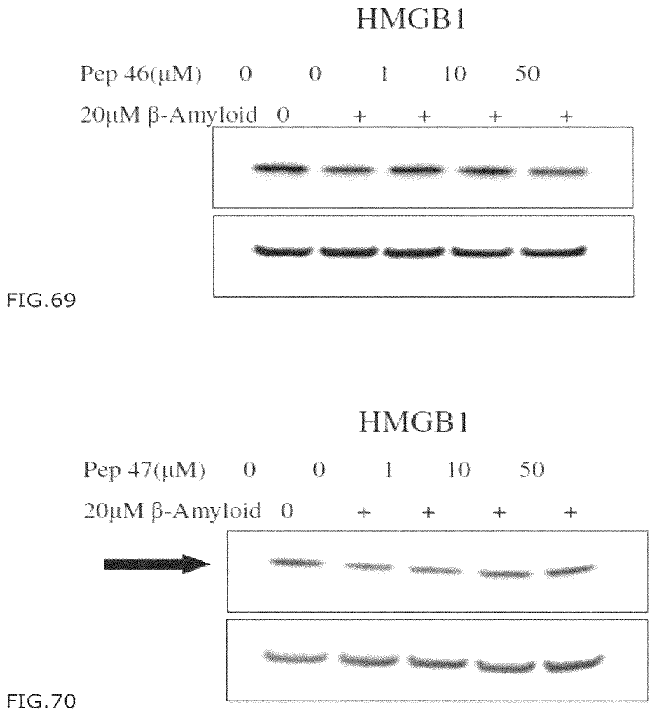

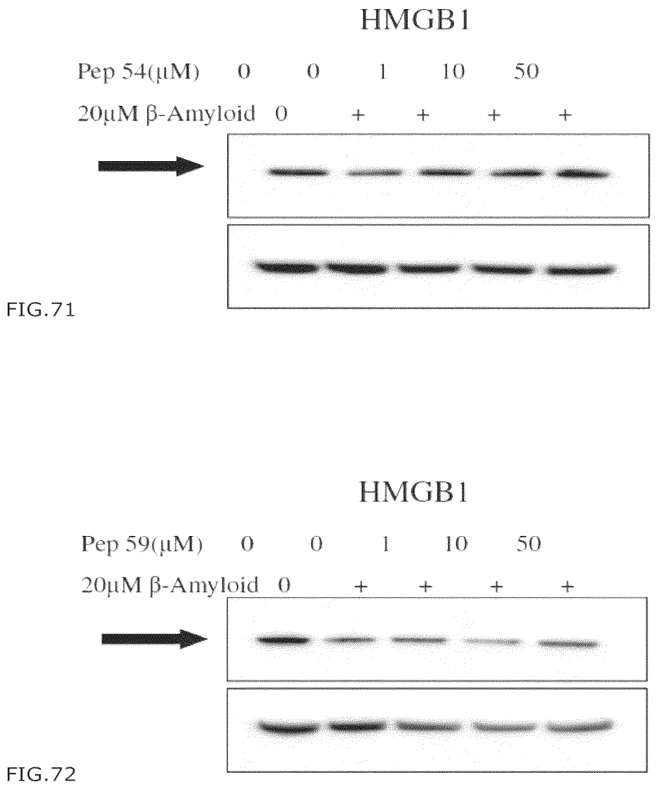

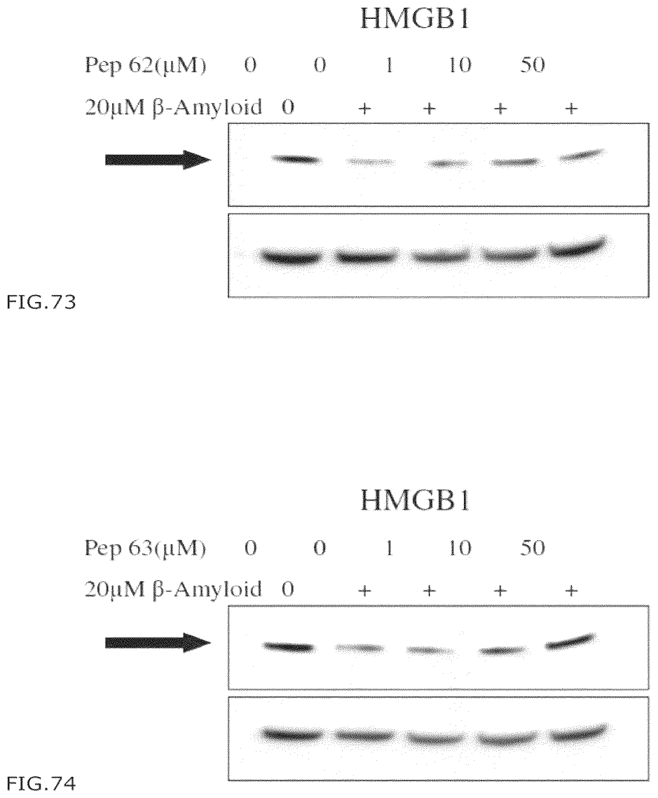

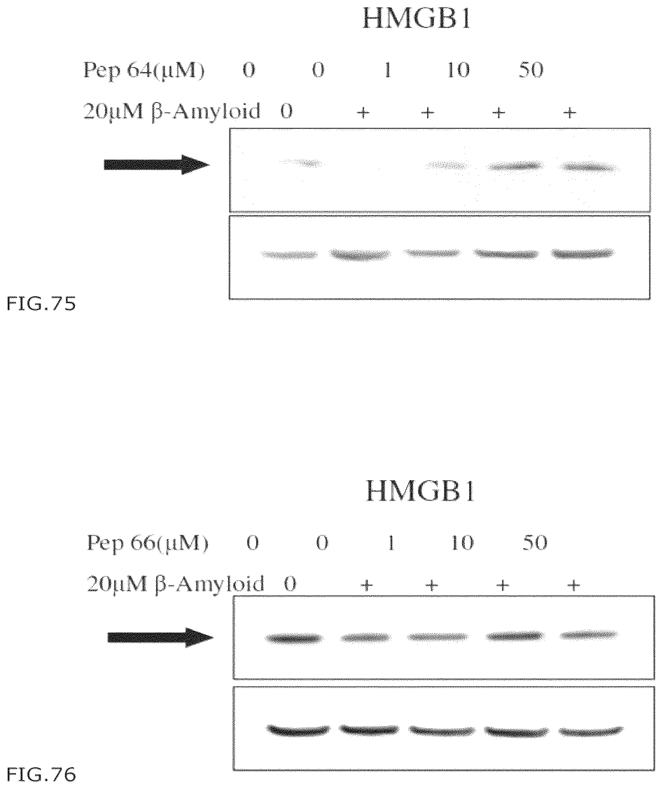

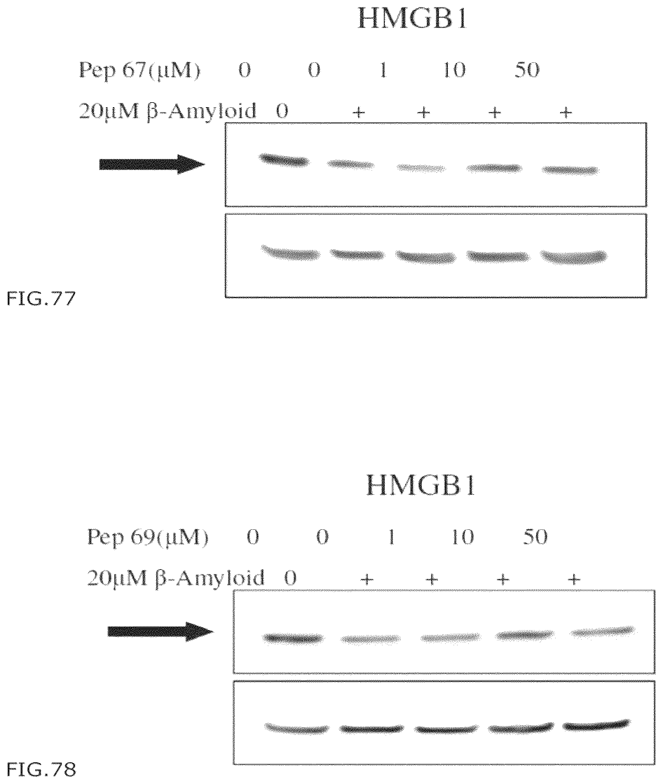

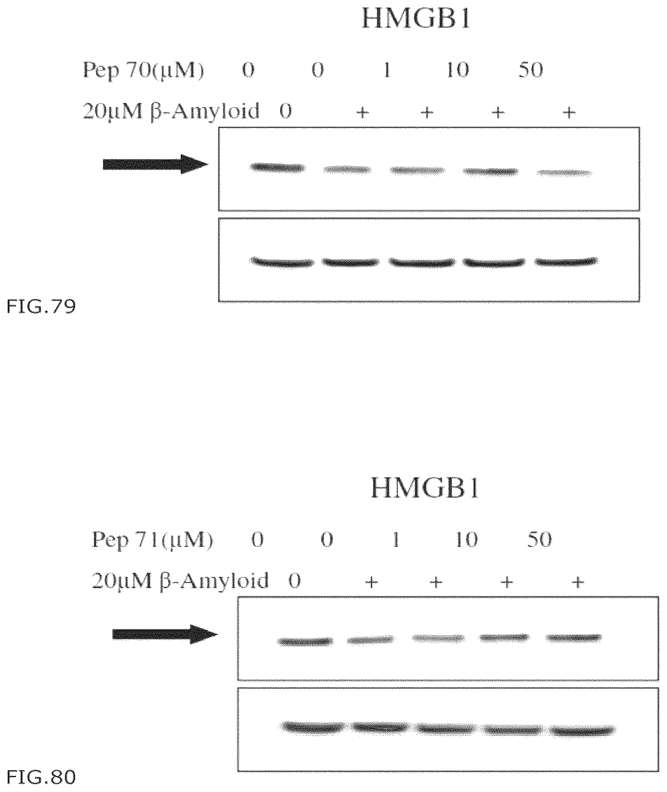

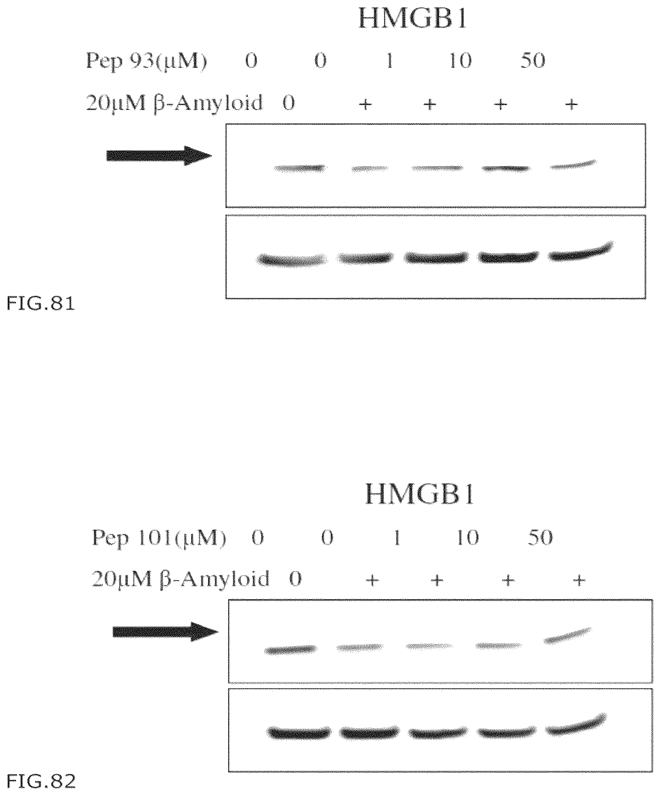

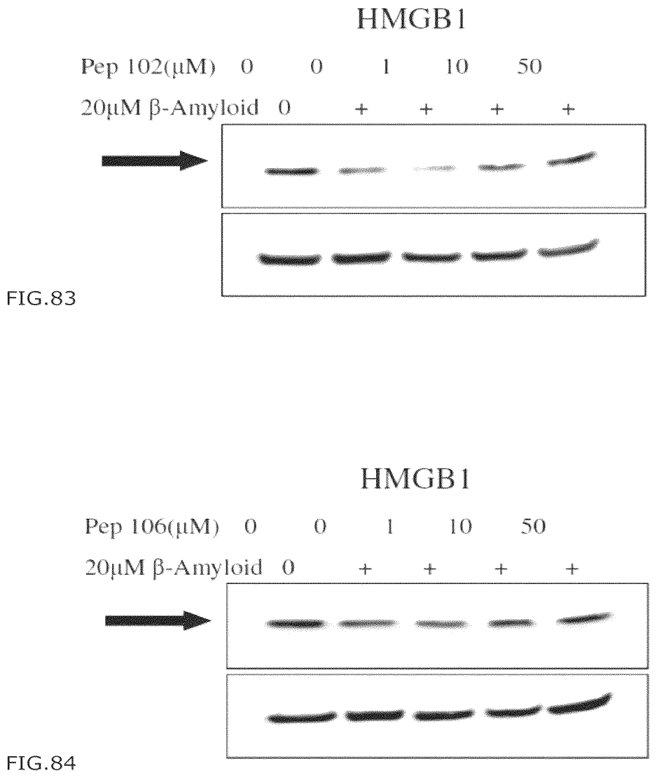

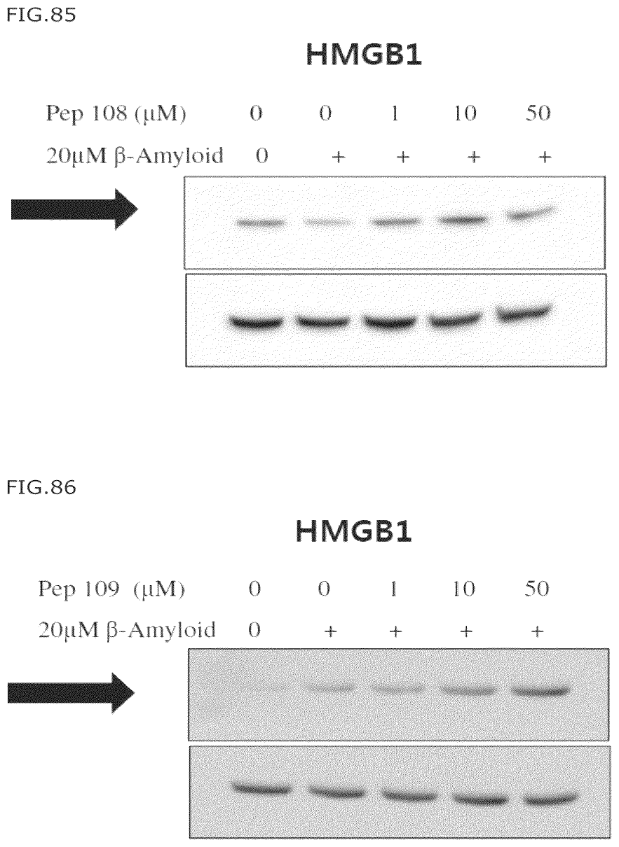

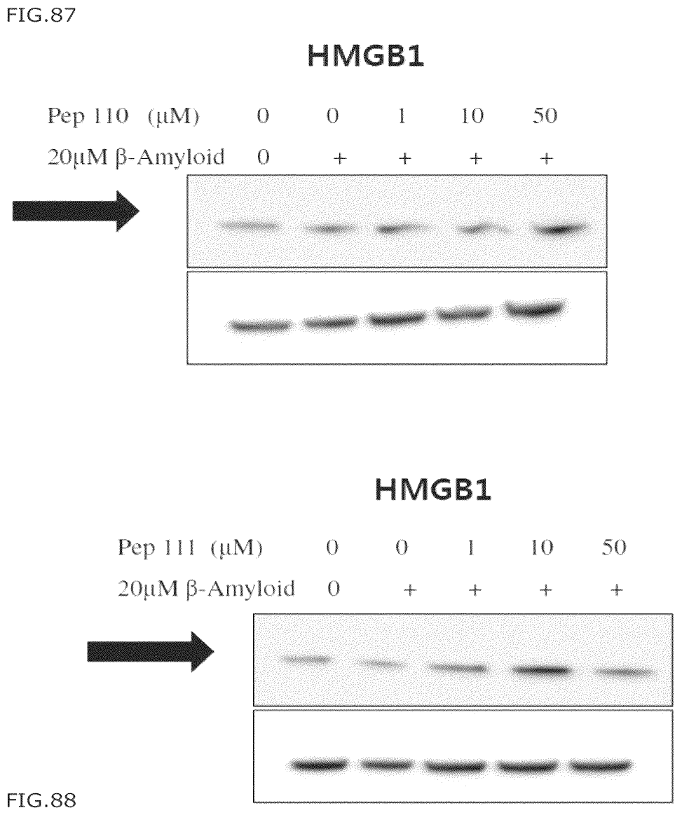

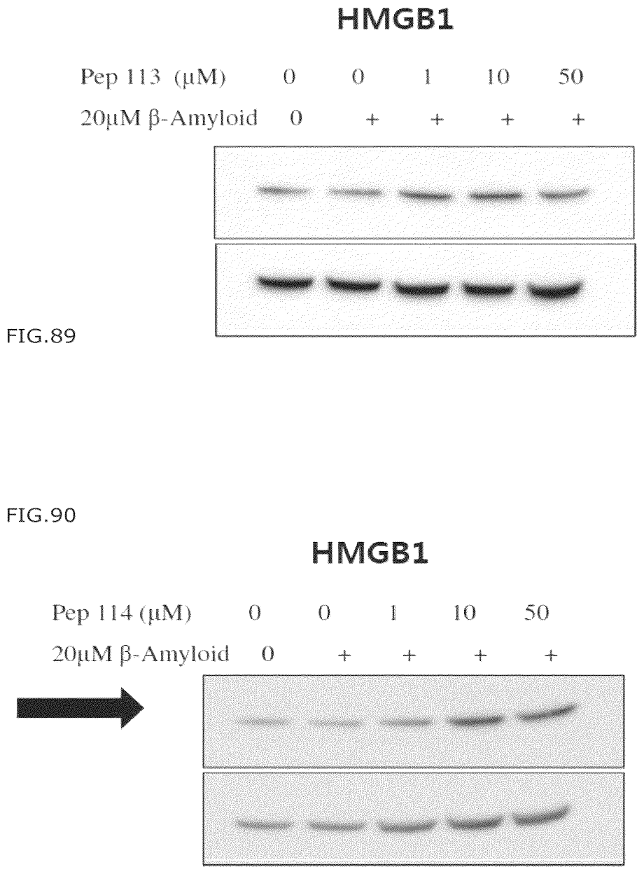

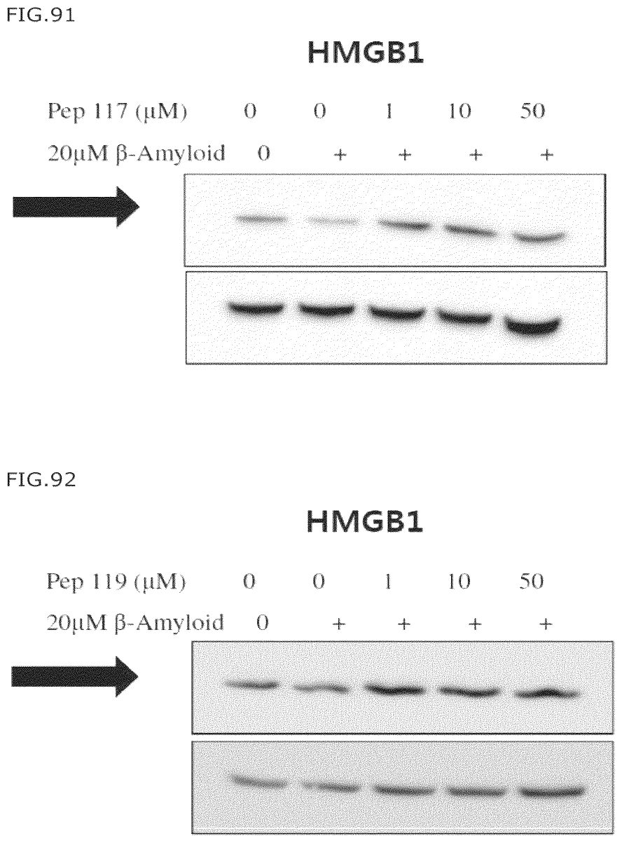

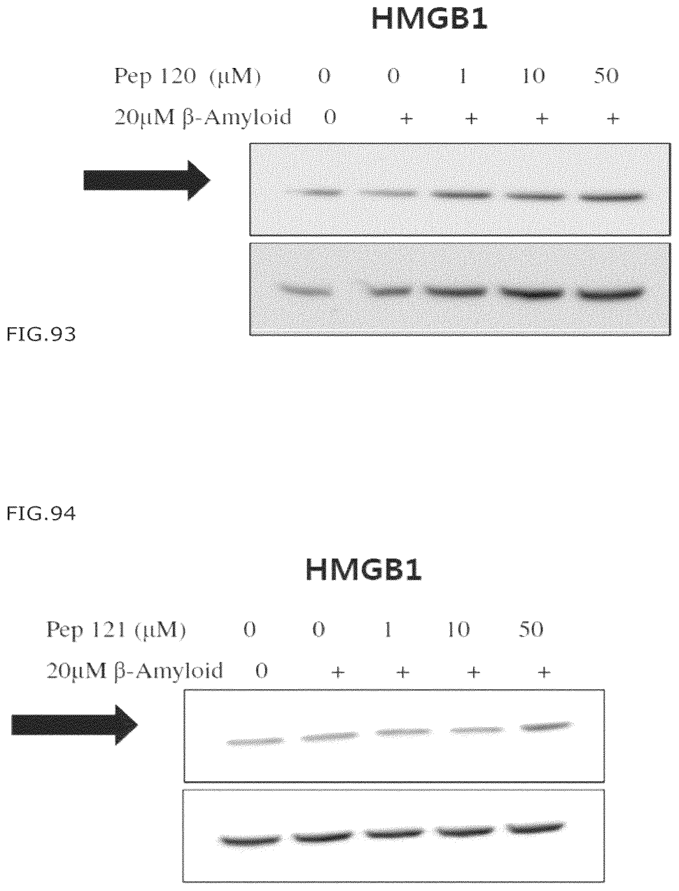

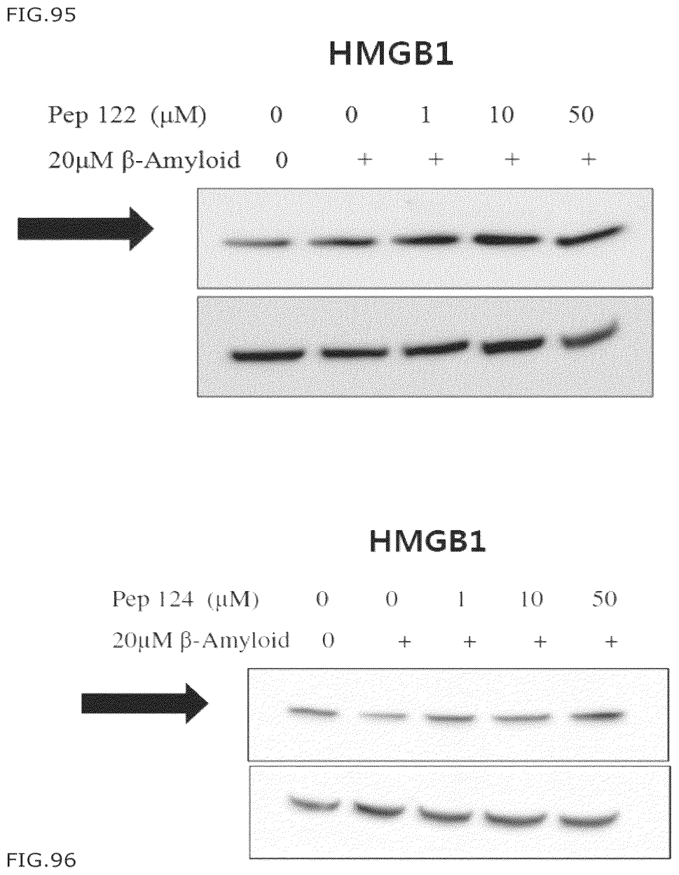

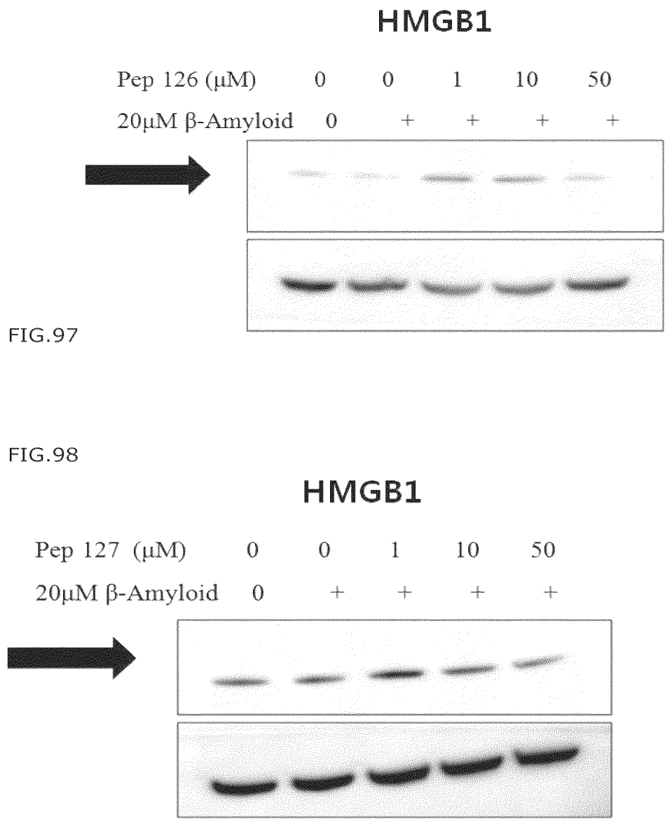

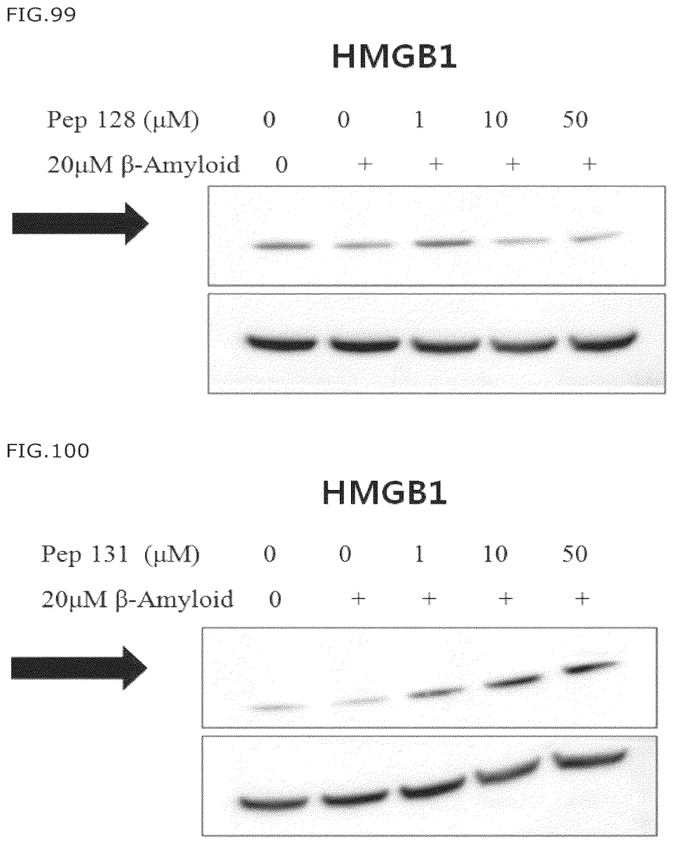

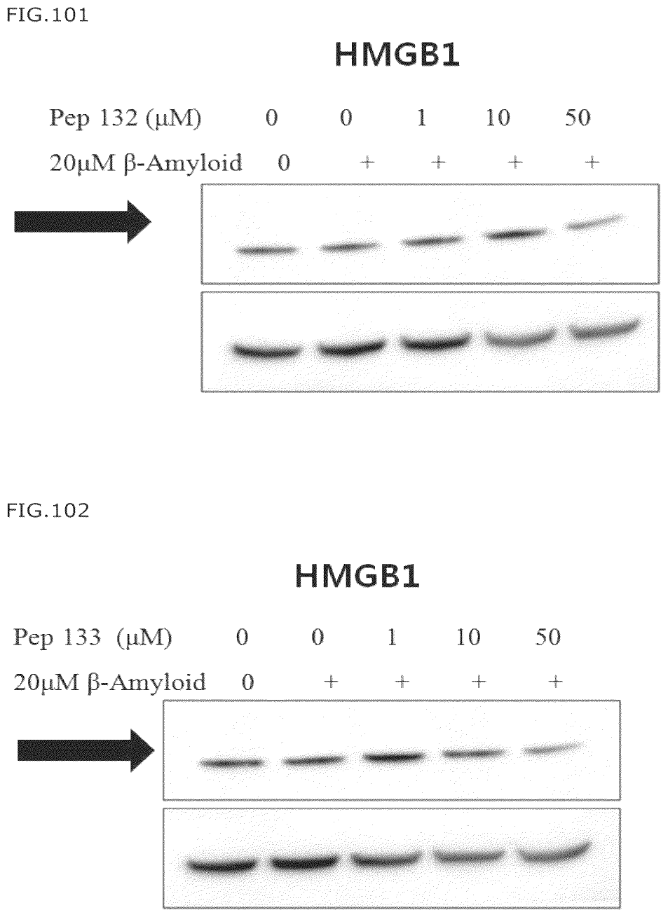

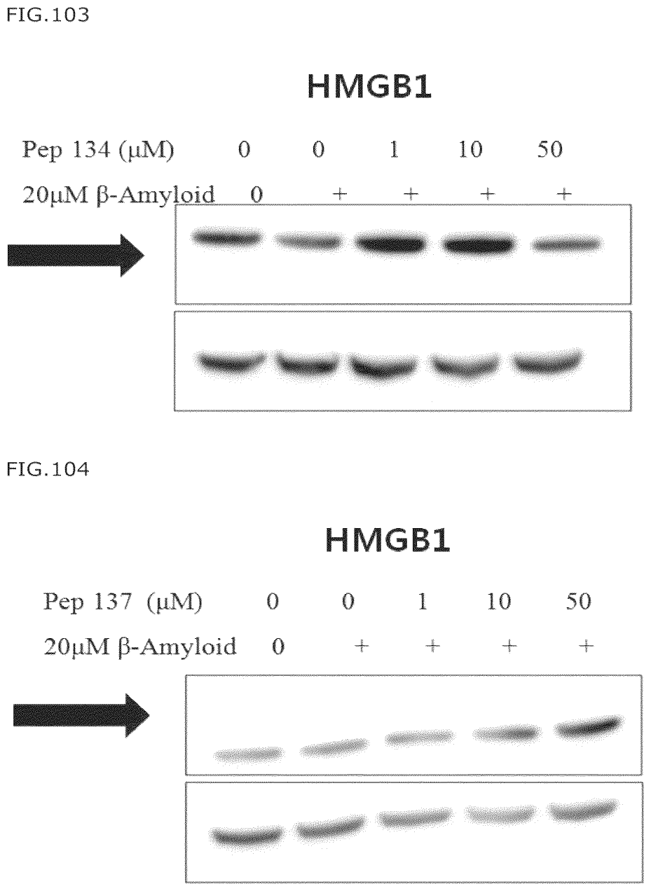

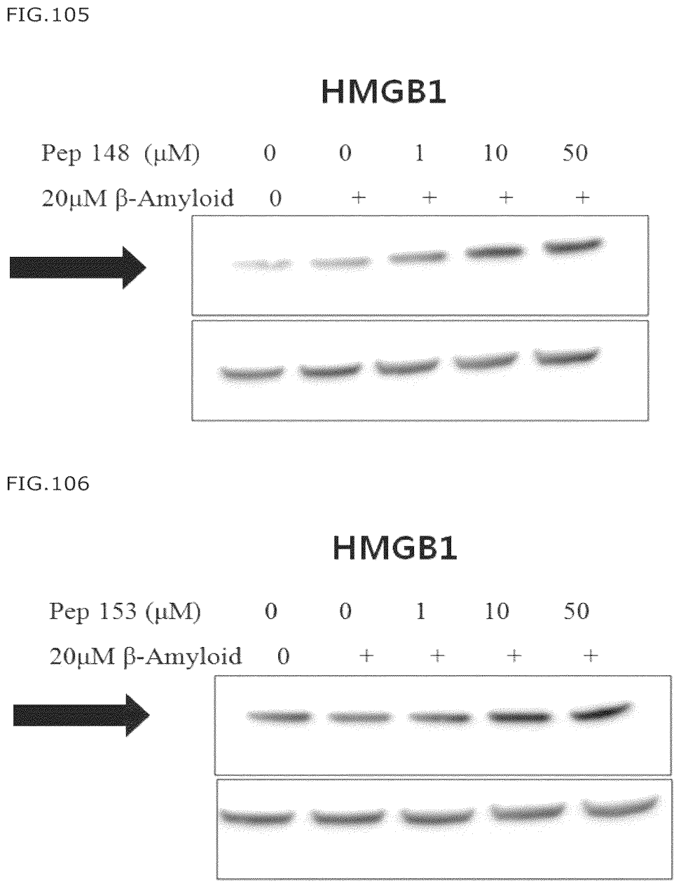

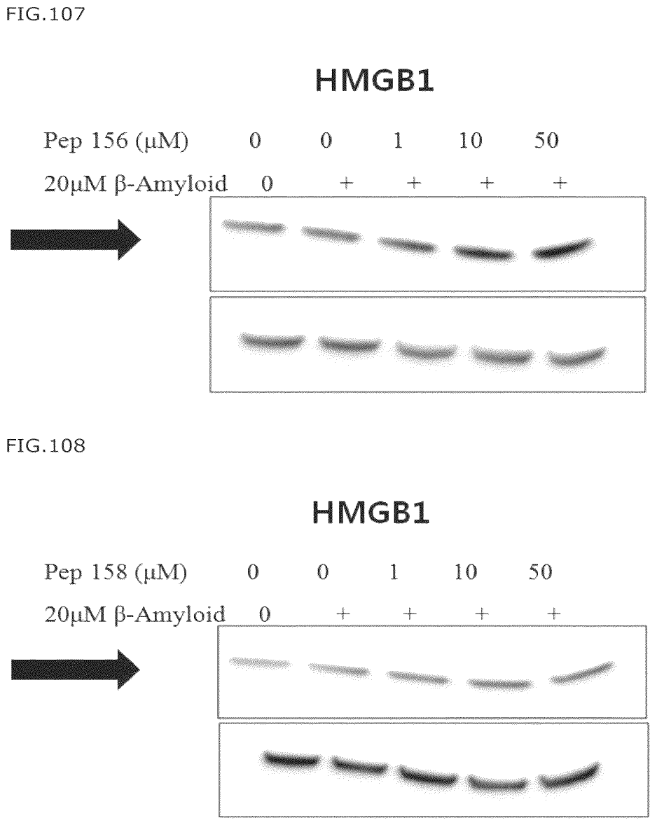

As a result of the western blot analysis, peptides showing accumulation of HMGβ1 in the cell were selected. FIG. 36 to FIG. 108 are the western blot results of selected peptides. Tubulins in these figures are used for confirming protein expression. The sequences of the selected peptides are as follows:

SEQ ID NO: 1, SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 12, SEQ ID NO: 14, SEQ ID NO: 15, SEQ ID NO: 17, SEQ ID NO: 18, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 33, SEQ ID NO: 34, SEQ ID NO: 35, SEQ 10 NO: 36, SEQ ID NO: 37, SEQ ID NO: 38, SEQ ID NO: 39, SEQ ID NO: 40, SEQ ID NO: 41, SEQ ID NO: 42, SEQ ID NO: 44, SEQ ID NO: 45, SEQ ID NO: 52, SEQ ID NO: 57, SEQ ID NO: 60, SEQ ID NO: 61, SEQ ID NO: 62, SEQ ID NO: 64, SEQ ID NO: 65, SEQ ID NO: 67, SEQ ID NO: 68, SEQ ID NO: 69, SEQ ID NO: 91, SEQ ID NO: 99, SEQ ID NO: 100, SEQ ID NO: 104, SEQ 10 NO: 106, SEQ ID NO: 107, SEQ ID NO: 108, SEQ ID NO: 109, SEQ ID NO: 111, SEQ ID NO: 112, SEQ ID NO: 115, SEQ ID NO: 117, SEQ ID NO: 118, SEQ ID NO: 119, SEQ ID NO: 120, SEQ ID NO: 122, SEQ ID NO: 124, SEQ ID NO: 125, SEQ ID NO: 126, SEQ ID NO: 129, SEQ ID NO: 130, SEQ ID NO: 131, SEQ 10 NO: 132, SEQ ID NO: 135, SEQ ID NO: 146, SEQ ID NO: 151, SEQ ID NO: 154, and SEQ ID NO: 156.

1-21. (canceled)

22. A peptide with anti-inflammatory activity, wherein the peptide consists of an amino acid sequence of SEQ ID NO: 108 or SEQ ID NO: 51.

23. The peptide according to claim 22, which originates from human telomerase.

24. A polynucleotide encoding a peptide according to claim 22.

25. An anti-inflammatory composition comprising the peptide according to claim 22 as an effective ingredient.

26. A method of treating or preventing inflammatory disease in a patient, comprising administering to the patient an isolated peptide having anti-inflammatory activity, wherein the peptide consists of the amino acid sequence of SEQ ID NO: 108 or SEQ ID NO: 51.

27. The method according to claim 26, wherein the inflammatory disease is selected from the group consisting of general or localized inflammatory disease; gastro-intestinal related diseases; dermal related diseases; vascular related disease; respiratory diseases; bone, joint, muscle, and connective tissue related diseases; urogenital disorders; central or peripheral nervous system related diseases; virus; and autoimmune diseases.

28. The method according to claim 26, wherein the daily dose of the peptide is 0.1 μg/kg/day to 1 g/kg/day.

29. A method of treating or preventing skin inflammation comprising administering an isolated peptide having anti-inflammatory activity, wherein the peptide consists of the amino acid sequence of SEQ ID NO: 108 or SEQ ID NO: 51.

30. The method according to claim 29, wherein the daily dose of the peptide is 0.1 μg/kg/day to 1 g/kg/day.

31. The method according to claim 27, wherein the central or peripheral nervous system related disease is selected from the group consisting of Alzheimer's disease; meningitis; encephalitis; multiple sclerosis; cerebral infarction; cerebral embolism; Guillain-Barre syndrome; neuritis; neuralgia; spinal cord injury; paralysis; and uveitis.


SEQUENCE LISTING (163 sequences)

SEQ ID NO: 1 - protein, 30 aa, Homo sapiens
ALFSVLNYERARRPGLLGASVLGLDDIHRA

SEQ ID NO: 2 - protein, 15 aa, Homo sapiens
SVLNYERARRPGLLG

SEQ ID NO: 3 - protein, 10 aa, Homo sapiens
GLLGASVLGL

SEQ ID NO: 4 - protein, 10 aa, Homo sapiens
ALLTSRLRFI

SEQ ID NO: 5 - protein, 9 aa, Homo sapiens
RPALLTSRL

SEQ ID NO: 6 - protein, 9 aa, Homo sapiens
RLTSRVKAL

SEQ ID NO: 7 - protein, 15 aa, Homo sapiens
RTFVLRVRAQDPPPE

SEQ ID NO: 8 - protein, 15 aa, Homo sapiens
RLTSRVKALFSVLNY

SEQ ID NO: 9 - protein, 15 aa, Homo sapiens
AERLTSRVKALFSVL

SEQ ID NO: 10 - protein, 9 aa, Homo sapiens
YERARRPGL

SEQ ID NO: 11 - protein, 9 aa, Homo sapiens
LLGASVLGL

SEQ ID NO: 12 - protein, 10 aa, Homo sapiens
VLGLDDIHRA

SEQ ID NO: 13 - protein, 10 aa, Homo sapiens
GASVLGLDDI

SEQ ID NO: 14 - protein, 10 aa, Homo sapiens
ALFSVLNYER

SEQ ID NO: 15 - protein, 10 aa, Homo sapiens
SVLNYERARR

SEQ ID NO: 16 - protein, 10 aa, Homo sapiens
SVLGLDDIHR

SEQ ID NO: 17 - protein, 10 aa, Homo sapiens
FSVLNYERAR

SEQ ID NO: 18 - protein, 10 aa, Homo sapiens
NYERARRPGL

SEQ ID NO: 19 - protein, 10 aa, Homo sapiens
YERARRPGLL

SEQ ID NO: 20 - protein, 10 aa, Homo sapiens
RPGLLGASVL

SEQ ID NO: 21 - protein, 9 aa, Homo sapiens
ERARRPGLL

SEQ ID NO: 22 - protein, 9 aa, Homo sapiens
VLGLDDIHR

SEQ ID NO: 23 - protein, 9 aa, Homo sapiens
SVLNYERAR

SEQ ID NO: 24 - protein, 9 aa, Homo sapiens
VLNYERARR

SEQ ID NO: 25 - protein, 9 aa, Homo sapiens
ARRPGLLGA

SEQ ID NO: 26 - protein, 9 aa, Homo sapiens
PGLLGASVL

SEQ ID NO: 27 - protein, 9 aa, Homo sapiens
RRPGLLGAS

SEQ ID NO: 28 - protein, 9 aa, Homo sapiens
ALFSVLNYE

SEQ ID NO: 29 - protein, 9 aa, Homo sapiens
GLLGASVLG

SEQ ID NO: 30 - protein, 9 aa, Homo sapiens
SVLGLDDIH

SEQ ID NO: 31 - protein, 8 aa, Homo sapiens
ERARRPGL

SEQ ID NO: 32 - protein, 8 aa, Homo sapiens
ARRPGLLG

SEQ ID NO: 33 - protein, 8 aa, Homo sapiens
GLLGASVL

SEQ ID NO: 34 - protein, 8 aa, Homo sapiens
RARRPGLL

SEQ ID NO: 35 - protein, 8 aa, Homo sapiens
LGASVLGL

SEQ ID NO: 36 - protein, 15 aa, Homo sapiens
VTETTFQKNRLFFYR

SEQ ID NO: 37 - protein, 15 aa, Homo sapiens
LFFYRKSVWSKLQSI

SEQ ID NO: 38 - protein, 15 aa, Homo sapiens
KLQSIGIRQHLKRVQ

SEQ ID NO: 39 - protein, 15 aa, Homo sapiens
EAEVRQHREARPALL

SEQ ID NO: 40 - protein, 15 aa, Homo sapiens
RPALLTSRLRFIPKP

SEQ ID NO: 41 - protein, 15 aa, Homo sapiens
FIPKPDGLRPIVNMD

SEQ ID NO: 42 - protein, 15 aa, Homo sapiens
RTFRREKRAERLTSR

SEQ ID NO: 43 - protein, 15 aa, Homo sapiens
LDDIHRAWRTFVLRV

SEQ ID NO: 44 - protein, 15 aa, Homo sapiens
FVLRVRAQDPPPELY

SEQ ID NO: 45 - protein, 15 aa, Homo sapiens
PQDRLTEVIASIIKP

SEQ ID NO: 46 - protein, 15 aa, Homo sapiens
KSVWSKLQSIGIRQH

SEQ ID NO: 47 - protein, 16 aa, Homo sapiens
LKRVQLRELSEAEVRQ

SEQ ID NO: 48 - protein, 20 aa, Homo sapiens
MPRAPRCRAVRSLLRSHYRE

SEQ ID NO: 49 - protein, 20 aa, Homo sapiens
VLPLATFVRRLGPQGWRLVQ

SEQ ID NO: 50 - protein, 20 aa, Homo sapiens
RGDPAAFRALVAQCLVCVPW

SEQ ID NO: 51 - protein, 20 aa, Homo sapiens
DARPPPAAPSFRQVSCLKEL

SEQ ID NO: 52 - protein, 20 aa, Homo sapiens
VARVLQRLCERGAKNVLAFG

SEQ ID NO: 53 - protein, 20 aa, Homo sapiens
FALLDGARGGPPEAFTTSVR

SEQ ID NO: 54 - protein, 20 aa, Homo sapiens
SYLPNTVTDALRGSGAWGLL

SEQ ID NO: 55 - protein, 20 aa, Homo sapiens
LRRVGDDVLVHLLARCALFV

SEQ ID NO: 56 - protein, 20 aa, Homo sapiens
LVAPSCAYQVCGPPLYQLGA

SEQ ID NO: 57 - protein, 20 aa, Homo sapiens
ATQARPPPHASGPRRRLGCE

SEQ ID NO: 58 - protein, 20 aa, Homo sapiens
RAWNHSVREAGVPLGLPAPG

SEQ ID NO: 59 - protein, 20 aa, Homo sapiens
ARRRGGSASRSLPLPKRPRR

SEQ ID NO: 60 - protein, 20 aa, Homo sapiens
GAAPEPERTPVGQGSWAHPG

SEQ ID NO: 61 - protein, 20 aa, Homo sapiens
RTRGPSDRGFCVVSPARPAE

SEQ ID NO: 62 - protein, 20 aa, Homo sapiens
EATSLEGALSGTRHSHPSVG

SEQ ID NO: 63 - protein, 20 aa, Homo sapiens
RQHHAGPPSTSRPPRPWDTP

SEQ ID NO: 64 - protein, 20 aa, Homo sapiens
CPPVYAETKHFLYSSGDKEQ

SEQ ID NO: 65 - protein, 20 aa, Homo sapiens
LRPSFLLSSLRPSLTGARRL

SEQ ID NO: 66 - protein, 20 aa, Homo sapiens
VETIFLGSRPWMPGTPRRLP

SEQ ID NO: 67 - protein, 20 aa, Homo sapiens
RLPQRYWQMRPLFLELLGNH

SEQ ID NO: 68 - protein, 20 aa, Homo sapiens
AQCPYGVLLKTHCPLRAAVT

SEQ ID NO: 69 - protein, 20 aa, Homo sapiens
PAAGVCAREKPQGSVAAPEE

SEQ ID NO: 70 - protein, 20 aa, Homo sapiens
EDTDPRRLVQLLRQHSSPWQ

SEQ ID NO: 71 - protein, 20 aa, Homo sapiens
VYGFVRACLRRLVPPGLWGS

SEQ ID NO: 72 - protein, 20 aa, Homo sapiens
RHNERRFLRNTKKFISLGKH

SEQ ID NO: 73 - protein, 20 aa, Homo sapiens
AKLSLQELTWKMSVRDCAWL

SEQ ID NO: 74 - protein, 20 aa, Homo sapiens
RRSPGVGCVPAAEHRLREEI

SEQ ID NO: 75 - protein, 20 aa, Homo sapiens
LAKFLHWLMSVYVVELLRSF

SEQ ID NO: 76 - protein, 20 aa, Homo sapiens
FYVTETTFQKNRLFFYRKSV

SEQ ID NO: 77 - protein, 20 aa, Homo sapiens
WSKLQSIGIRQHLKRVQLRE

SEQ ID NO: 78 - protein, 20 aa, Homo sapiens
LSEAEVRQHREARPALLTSR

SEQ ID NO: 79 - protein, 20 aa, Homo sapiens
LRFIPKPDGLRPIVNMDYVV

SEQ ID NO: 80 - protein, 20 aa, Homo sapiens
GARTFRREKRAERLTSRVKA

SEQ ID NO: 81 - protein, 20 aa, Homo sapiens
LFSVLNYERARRPGLLGASV

SEQ ID NO: 82 - protein, 20 aa, Homo sapiens
LGLDDIHRAWRTFVLRVRAQ

SEQ ID NO: 83 - protein, 20 aa, Homo sapiens
DPPPELYFVKVDVTGAYDTI

SEQ ID NO: 84 - protein, 20 aa, Homo sapiens
PQDRLTEVIASIIKPQNTYC

SEQ ID NO: 85 - protein, 20 aa, Homo sapiens
VRRYAVVQKAAHGHVRKAFK

SEQ ID NO: 86 - protein, 20 aa, Homo sapiens
SHVSTLTDLQPYMRQFVAHL

SEQ ID NO: 87 - protein, 20 aa, Homo sapiens
QETSPLRDAVVIEQSSSLNE

SEQ ID NO: 88 - protein, 20 aa, Homo sapiens
ASSGLFDVFLRFMCHHAVRI

SEQ ID NO: 89 - protein, 20 aa, Homo sapiens
RGKSYVQCQGIPQGSILSTL

SEQ ID NO: 90 - protein, 20 aa, Homo sapiens
LCSLCYGDMENKLFAGIRRD

SEQ ID NO: 91 - protein, 20 aa, Homo sapiens
GLLLRLVDDFLLVTPHLTHA

SEQ ID NO: 92 - protein, 20 aa, Homo sapiens
KTFLRTLVRGVPEYGCVVNL

SEQ ID NO: 93 - protein, 20 aa, Homo sapiens
RKTVVNFPVEDEALGGTAFV

SEQ ID NO: 94 - protein, 20 aa, Homo sapiens
QMPAHGLFPWCGLLLDTRTL

SEQ ID NO: 95 - protein, 20 aa, Homo sapiens
EVQSDYSSYARTSIRASLTF

SEQ ID NO: 96 - protein, 20 aa, Homo sapiens
NRGFKAGRNMRRKLFGVLRL

SEQ ID NO: 97 - protein, 20 aa, Homo sapiens
KCHSLFLDLQVNSLQTVCTN

SEQ ID NO: 98 - protein, 20 aa, Homo sapiens
IYKILLLQAYRFHACVLQLP

SEQ ID NO: 99 - protein, 20 aa, Homo sapiens
FHQQVWKNPTFFLRVISDTA

SEQ ID NO: 100 - protein, 20 aa, Homo sapiens
SLCYSILKAKNAGMSLGAKG

SEQ ID NO: 101 - protein, 20 aa, Homo sapiens
AAGPLPSEAVQWLCHQAFLL

SEQ ID NO: 102 - protein, 20 aa, Homo sapiens
KLTRHRVTYVPLLGSLRTAQ

SEQ ID NO: 103 - protein, 20 aa, Homo sapiens
TQLSRKLPGTTLTALEAAAN

SEQ ID NO: 104 - protein, 12 aa, Homo sapiens
PALPSDFKTILD

SEQ ID NO: 105 - protein, 10 aa, Homo sapiens
MPRAPRCRAV

SEQ ID NO: 106 - protein, 20 aa, Homo sapiens
RSLLRSHYREVLPLATFVRR

SEQ ID NO: 107 - protein, 20 aa, Homo sapiens
LGPQGWRLVQRGDPAAFRAL

SEQ ID NO: 108 - protein, 20 aa, Homo sapiens
VAQCLVCVPWDARPPPAAPS

SEQ ID NO: 109 - protein, 20 aa, Homo sapiens
FRQVSCLKELVARVLQRLCE

SEQ ID NO: 110 - protein, 20 aa, Homo sapiens
RGAKNVLAFGFALLDGARGG

SEQ ID NO: 111 - protein, 20 aa, Homo sapiens
PPEAFTTSVRSYLPNTVTDA

SEQ ID NO: 112 - protein, 20 aa, Homo sapiens
LRGSGAWGLLLRRVGDDVLV

SEQ ID NO: 113 - protein, 20 aa, Homo sapiens
HLLARCALFVLVAPSCAYQV

SEQ ID NO: 114 - protein, 20 aa, Homo sapiens
CGPPLYQLGAATQARPPPHA

SEQ ID NO: 115 - protein, 20 aa, Homo sapiens
SGPRRRLGCERAWNHSVREA

SEQ ID NO: 116 - protein, 20 aa, Homo sapiens
GVPLGLPAPGARRRGGSASR

SEQ ID NO: 117 - protein, 20 aa, Homo sapiens
SLPLPKRPRRGAAPEPERTP

SEQ ID NO: 118 - protein, 20 aa, Homo sapiens
VGQGSWAHPGRTRGPSDRGF

SEQ ID NO: 119 - protein, 20 aa, Homo sapiens
CVVSPARPAEEATSLEGALS

SEQ ID NO: 120 - protein, 20 aa, Homo sapiens
GTRHSHPSVGRQHHAGPPST

SEQ ID NO: 121 - protein, 20 aa, Homo sapiens
SRPPRPWDTPCPPVYAETKH

SEQ ID NO: 122 - protein, 20 aa, Homo sapiens
FLYSSGDKEQLRPSFLLSSL

SEQ ID NO: 123 - protein, 20 aa, Homo sapiens
RPSLTGARRLVETIFLGSRP

SEQ ID NO: 124 - protein, 20 aa, Homo sapiens
WMPGTPRRLPRLPQRYWQMR

SEQ ID NO: 125 - protein, 20 aa, Homo sapiens
PLFLELLGNHAQCPYGVLLK

SEQ ID NO: 126 - protein, 20 aa, Homo sapiens
THCPLRAAVTPAAGVCAREK

SEQ ID NO: 127 - protein, 20 aa, Homo sapiens
PQGSVAAPEEEDTDPRRLVQ

SEQ ID NO: 128 - protein, 20 aa, Homo sapiens
LLRQHSSPWQVYGFVRACLR

SEQ ID NO: 129 - protein, 20 aa, Homo sapiens
RLVPPGLWGSRHNERRFLRN

SEQ ID NO: 130 - protein, 20 aa, Homo sapiens
TKKFISLGKHAKLSLQELTW

SEQ ID NO: 131 - protein, 20 aa, Homo sapiens
KMSVRDCAWLRRSPGVGCVP

SEQ ID NO: 132 - protein, 20 aa, Homo sapiens
AAEHRLREEILAKFLHWLMS

SEQ ID NO: 133 - protein, 20 aa, Homo sapiens
VYVVELLRSFFYVTETTFQK

SEQ ID NO: 134 - protein, 20 aa, Homo sapiens
NRLFFYRKSVWSKLQSIGIR

SEQ ID NO: 135 - protein, 20 aa, Homo sapiens
QHLKRVQLRELSEAEVRQHR

SEQ ID NO: 136 - protein, 20 aa, Homo sapiens
EARPALLTSRLRFIPKPDGL

SEQ ID NO: 137 - protein, 20 aa, Homo sapiens
RPIVNMDYVVGARTFRREKR

SEQ ID NO: 138 - protein, 20 aa, Homo sapiens
AERLTSRVKALFSVLNYERA

SEQ ID NO: 139 - protein, 20 aa, Homo sapiens
RRPGLLGASVLGLDDIHRAW

SEQ ID NO: 140 - protein, 20 aa, Homo sapiens
RTFVLRVRAQDPPPELYFVK

SEQ ID NO: 141 - protein, 20 aa, Homo sapiens
VDVTGAYDTIPQDRLTEVIA

SEQ ID NO: 142 - protein, 20 aa, Homo sapiens
SIIKPQNTYCVRRYAVVQKA

SEQ ID NO: 143 - protein, 20 aa, Homo sapiens
AHGHVRKAFKSHVSTLTDLQ

SEQ ID NO: 144 - protein, 20 aa, Homo sapiens
PYMRQFVAHLQETSPLRDAV

SEQ ID NO: 145 - protein, 20 aa, Homo sapiens
VIEQSSSLNEASSGLFDVFL

SEQ ID NO: 146 - protein, 20 aa, Homo sapiens
RFMCHHAVRIRGKSYVQCQG

SEQ ID NO: 147 - protein, 20 aa, Homo sapiens
IPQGSILSTLLCSLCYGDME

SEQ ID NO: 148 - protein, 20 aa, Homo sapiens
NKLFAGIRRDGLLLRLVDDF

SEQ ID NO: 149 - protein, 20 aa, Homo sapiens
LLVTPHLTHAKTFLRTLVRG

SEQ ID NO: 150 - protein, 20 aa, Homo sapiens
VPEYGCVVNLRKTVVNFPVE

SEQ ID NO: 151 - protein, 20 aa, Homo sapiens
DEALGGTAFVQMPAHGLFPW

SEQ ID NO: 152 - protein, 20 aa, Homo sapiens
CGLLLDTRTLEVQSDYSSYA

SEQ ID NO: 153 - protein, 20 aa, Homo sapiens
RTSIRASLTFNRGFKAGRNM

SEQ ID NO: 154 - protein, 20 aa, Homo sapiens
RRKLFGVLRLKCHSLFLDLQ

SEQ ID NO: 155 - protein, 20 aa, Homo sapiens
VNSLQTVCTNIYKILLLQAY

SEQ ID NO: 156 - protein, 20 aa, Homo sapiens
RFHACVLQLPFHQQVWKNPT

SEQ ID NO: 157 - protein, 20 aa, Homo sapiens
FFLRVISDTASLCYSILKAK

SEQ ID NO: 158 - protein, 20 aa, Homo sapiens
NAGMSLGAKGAAGPLPSEAV

SEQ ID NO: 159 - protein, 20 aa, Homo sapiens
QWLCHQAFLLKLTRHRVTYV

SEQ ID NO: 160 - protein, 20 aa, Homo sapiens
PLLGSLRTAQTQLSRKLPGT

SEQ ID NO: 161 - protein, 22 aa, Homo sapiens
TLTALEAAANPALPSDFKTILD

SEQ ID NO: 162 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 163 - protein, 16 aa, Homo sapiens
EARPALLTSRLRFIPK